(19) United States
(12) Patent Application Publication
(10) Pub. No.: US 2019/0125896 A1
(43) Pub. Date: May 2, 2019

(54) ANTI-INFECTIVE COMPOSITIONS COMPRISING PHYTOGLYCOGEN NANOPARTICLES

(71) Applicant: MIREXUS BIOTECHNOLOGIES INC., Guelph (CA)

(72) Inventors: Sarah Ruth SCHOOLING, Guelph (CA); Lisa Suzanne BERTOLO, Guelph (CA); Anton KORENEVSKI, Guelph (CA); Erzsebet PAPP-SZABO, Guelph (CA); Karl Michael KLINGER, Guelph (CA)

(73) Assignee: MIREXUS BIOTECHNOLOGIES INC., Guelph, ON (CA)

(21) Appl. No.: 16/093,389

(22) PCT Filed: Apr. 18, 2017
(86) PCT No.: PCT/CA2017/050472

§ 371 Date: Oct. 12, 2018

Related U.S. Application Data
(60) Provisional application No. 62/322,478, filed on Apr. 14, 2016.

(51) Int. Cl. A61K 47/69; A61K 31/14; A61K 9/00; A61L 27/20; A61L 27/54; A61L 27/34; A61K 31/045; A01N 25/10; A01N 33/12

(52) U.S. Cl. CPC A61K 47/6939; A61K 31/14; A61K 9/0014; A61L 27/20; A61L 27/54; A61L 2400/12; A61K 31/045; A01N 25/10; A01N 33/12; A61L 2300/404; A61L 27/34

(57) ABSTRACT

An anti-infective composition comprising glycogen or phytoglycogen nanoparticles is provided.

### TECHNICAL FIELD

This invention relates to anti-infective compositions.

### BACKGROUND OF THE ART

Glycogen is a short-term energy storage material in animals. In mammals, glycogen occurs in muscle and liver tissues. It is comprised of 1,4-glucan chains, highly branched via at 6-glucosidic linkages with a molecular weight of 10⁶-10⁸ Daltons. Glycogen is present in animal tissues and is also found to accumulate in microorganisms, e.g., in bacteria and yeasts.

Phytoglycogen is a polysaccharide that is very similar to glycogen, both in terms of its structure and physical properties. It is distinguished from glycogen based on its plant-based sources of origin. The most prominent sources of phytoglycogen are kernels of sweet corn, as well as specific varieties of rice, barley, and sorghum.

Applications of glycogen, phytoglycogen and related glycogen-like material have been suggested.

### BRIEF SUMMARY

The present disclosure relates to anti-infective compositions comprising glycogen or phytoglycogen nanoparticles, including modified glycogen or phytoglycogen such as cationized phytoglycogen functionalized with quaternary ammonium compounds (herein referred to as a “phytoglycogen nanoparticle(s)”). Further, the present disclosure relates to compositions comprising phytoglycogen nanoparticles for use as anti-infectives.

In one embodiment, the phytoglycogen nanoparticles are functionalized with a primary, secondary, tertiary or quaternary ammonium compound. In a preferred embodiment, the phytoglycogen nanoparticles are functionalized with a quaternary ammonium compound. In one embodiment, the phytoglycogen nanoparticles are functionalized with a quaternary ammonium compound having the general structure:

P/G-(linker)-N(R₁R₂R₃)⁺

with R_{1/2/3} being C₁-C₃₂ alkyl chains. In some embodiments, R_{1/2/3} are C₁-C₃₀ alkyl chains, preferably C₁-C₂₄ alkyl chains. In other embodiments, the linker is optional and the quaternary ammonium compound is directly attached to the nanoparticle. The linker may comprise a C₁-C₃₂ alkyl chain with or without further functional groups, or an oligomer or polymer such as polyethylene oxide or polyethylene imine.

In one embodiment, the anti-infective composition comprises glycogen or phytoglycogen nanoparticles, with an anti-infective component, wherein the anti-infective component comprises one or more molecules that impart anti-infective activity to the composition, and a carrier.

In one embodiment, the anti-infective composition comprises a composition of monodisperse phytoglycogen nanoparticles having a polydispersity index (PDI) of less than about 0.3 as measured by dynamic light scattering. In one embodiment, the anti-infective composition comprises a composition of monodisperse phytoglycogen nanoparticles having an average particle diameter of between about 30 nm and about 150 nm. In one embodiment, the anti-infective composition comprises a composition of monodisperse phytoglycogen nanoparticles having an average particle diameter of about 60 nm to about 110 nm.

In one embodiment, the anti-infective component comprises an antibiotic, an antifungal, an anti-parasitic or an anti-protozoal compound.

In various embodiments, the phytoglycogen nanoparticles are conjugated to one or more of an antibiotic, an antifungal, an anti-parasite and/or anti-protozoal compound. In other embodiments, the phytoglycogen nanoparticles are administered concurrently with one or more of an antibiotic, an antifungal, an anti-parasite and/or anti-protozoal compound.

In one embodiment, the anti-infectives are used as a biofilm inhibitor. In one embodiment, the composition decreases or inhibits biofilm formation, maintenance or growth. In another embodiment, the composition interferes with quorum sensing processes and the production of virulence factors.

In some embodiments, the anti-infective composition can be used as skin sanitizer or surface sanitizer, wherein the sanitizer is in the form of a gel, lotion, wash or spray. In other embodiments, the anti-infective composition is used to treat an intracellular infection.

### BRIEF DESCRIPTION OF THE DRAWINGS

FIG. 1 shows phytoglycogen/glycogen nanoparticle derivatization via cyanylation.

FIG. 2 is a schematic drawing of a phytoglycogen/glycogen nanoparticle.

FIG. 3 shows the cytotoxicity as measured by dead cells due to monodisperse glycogen nanoparticles on Hep2 (cancer liver cells) as compared to poly(lactic-co-glycolic acid) (PLGA).

FIG. 4 shows the cytotoxicity as measured by release of lactate dehydrogenase (LDH) by monodisperse glycogen nanoparticles (nps) on Hep2 (cancer liver cells) as compared to poly(lactic-co-glycolic acid) (PLGA).

FIG. 5 shows fluorescence microscopy of normal murine endothelial cells incubated with monodisperse phytoglycogen nanoparticles conjugated to Rhodamine B.

FIG. 6 shows fluorescence microscopy of white blood cells incubated with monodisperse phytoglycogen nanoparticles conjugated with Rhodamine B.

FIG. 7 shows pyocyanin production by P. aeruginosa during growth in the presence or absence of native phytoglycogen (dark grey bars) and its cationized form (hollow bars). Data are the average of three independent experiments, with internal triplicate replicate (n=9±SEM).

FIG. 8 shows that (a) swimming (b) twitching and, (c) swarming motility of P. aeruginosa PAO1 is negatively affected by cationized phytoglycogen (□) but not native phytoglycogen (▪). Data are normalized relative to the average value obtained under non-supplemented conditions. Results were confirmed by independent triplicate experiments with in-assay triplicate replicate and three measurements recorded per plate (n=27±SEM).

FIG. 9 shows representative images of biofilms formed by P. aeruginosa in modified M9 medium supplemented with native or cationized phytoglycogen.

FIG. 10 shows quantification of biofilm accretion by P. aeruginosa in modified M9 medium (▪) or King's A medium (♦) supplemented with native phytoglycogen. Ratio data are the average of n=9±SEM; absorbance values were normalized to the average absorbance value of biofilm grown in medium only.

FIG. 11 shows the quantification of biofilm formation by P. aeruginosa in modified M9 medium (□) or King's A medium (⋄) supplemented with cationized phytoglycogen. Ratio data are the average of n=9±SEM; absorbance values were normalized to the average absorbance value of biofilm grown in medium only.

FIG. 12 shows representative images of biofilm accretion by P. aeruginosa following treatment of pre-formed biofilms with cationized phytoglycogen.

FIG. 13 shows the removal of pre-formed biofilms by P. aeruginosa following treatment with cationized phytoglycogen. Experiments were performed as quadruplicate in-assay replicates and were repeated three times. Data are normalized relative to the average A₅₇₀ obtained for the 20 hT biofilm subset (n=12±SEM).

FIG. 14 shows that short-term exposure of 20 h P. aeruginosa biofilms to cationized phytoglycogen causes a reduction in biofilm. 20 h biofilms were exposed to medium only (dark bars), and medium with 1 mg native phytoglycogen.ml⁻¹ (grey bars) or with 1 mg cationized phytoglycogen.ml⁻¹ (hollow bars). Values are the average of n=12±SEM.

FIG. 15 shows cationized phytoglycogen prevents the enhanced biofilm formation which is an undesirable feature of sub-MIC of select antibiotics. Absorbance data were normalized to the corresponding medium condition without antibiotic. Assays were done in Mueller-Hinton medium (●) or medium supplemented with 1 ( ) or 10 mg (◯) cationized phytoglycogen.ml⁻¹. Values are the average of n=12±SEM.

FIG. 16 shows that a combination of cationized phytoglycogen and the antibiotic tobramycin enhances biofilm eradication. Absorbance data were normalized to the corresponding medium condition without antibiotic. Assays were done in Mueller-Hinton medium (●) or medium supplemented with 1 ( ) or 10 mg (∘) cationized phytoglycogen.ml⁻¹. Values are the average of n=12±SEM.

FIG. 17 shows that a combination of cationized phytoglycogen and the antibiotic ciprofloxacin enhances biofilm eradication. Absorbance data were normalized to the corresponding medium condition without antibiotic. Assays were done in Mueller-Hinton medium (●) or medium supplemented with 1 ( ) or 10 mg (◯) cationized phytoglycogen.ml⁻¹. Values are the average of n=12±SEM.

FIG. 18 shows that cationized but not native phytoglycogen causes the sedimentation of cells from suspension. Representative images are presented of microfuge tubes containing suspensions of cells incubated in medium supplemented with native or cationized phytoglycogen. Note the formation of material (cells) at the bottom of the tube containing cationized phytoglycogen, which was accompanied by a concomitant clarification of the upper liquid phase.

FIG. 19 shows representative transmission electron micrographs of P. aeruginosa cells incubated with native or cationized phytoglycogen. The dark arrows indicate phytoglycogen. Note the localization of cationized phytoglycogen at the cell surface; white arrows indicate regions of cell surface perturbation. The scale bar represents 1 μm.

FIG. 20 shows the internalization of Cy5.5-labelled PHX particles by THP-1 monocytes: Fluorescence confocal images of THP-1 cells incubated with Cy5.5-Phytoglycogen nanoparticles (1 mg/mL) at 4° C. for 24 hrs (A), at 37° C. for 6 hrs (B) and at 37° C. for 24 hrs. Cy5.5-Phytoglycogen nanoparticles, Nucleus stained with DAPI, and cell membrane stained with AF488.

FIG. 21 shows the pharmacokinetic profile of Cy5.5-phytoglycogen taken from repeated blood sampling of nude CD-1 mice.

FIG. 22 shows the quantification of fluorescent signals in organs imaged ex vivo at 30 min and 24 hrs after i.v. injection in naïve nude CD-1 mice. The average fluorescence concentration data, suggests that in addition to the liver and kidney, high signal can also be detected in lung and heart. The fluorescence concentrations at 30 mins are higher than at 24 hrs. Pre-scan data indicates the fluorescence concentration data for a mouse not injected with Cy5.5-Phytoglycogen (i.e. background autofluorescence). Data are presented as mean+/−SD.

FIG. 23 shows the quantification of fluorescent signals in brain imaged ex vivo at 30 min and 24 hrs after i.v. injection of Cy5.5-Phytoglycogen in naïve nude CD-1 mice. The data indicate that compared to pre-scan (autofluorescence level), there are measurable signals in the brain from Cy5.5-Phytoglycogen. The signal is highest at 30 mins and goes down slowly over time at 24 hrs.

### DETAILED DESCRIPTION

As used herein, the term “anti-infective” refers to an agent that limits the progression or spread of infection. Anti-infectives include antimicrobials such as antibacterials, antifungals and antiparasitics, which act by limiting cell growth or causing cell death. Anti-infectives also include those agents which limit the progression or spread of infection though mechanisms other than growth inhibition and cell death. Anti-infectives may act by altering the physiological responses of both infectious agent and the target host. Quorum sensing inhibitors are an example of the former; vaccines of the latter. The term “anti-infective” as used herein may act through both antimicrobial activity, and also through the attenuation or modification of the production of virulence factors. The term “virulence factors” as used herein are those factors produced by a cell which contribute to that organism's capabilities to cause infection. Virulence factors may be excreted, secreted or shed from the cell (e.g. enzymes, toxins), may be part of the cell (e.g. membrane modifications), or a behaviour of the cell (e.g. motility, biofilm formation)

The terms “antibiotic” and “antibacterial” are used interchangeably to refer to agents used in the treatment or prevention of bacterial infection or the spread of bacteria, and include both agents that kill bacteria or inhibit the growth of bacteria. The term “antifungal” is used to refer to agents used in the treatment or prevention of fungal infection or the spread of fungi, and includes both agents that kill fungi or inhibit the growth of fungi.

As used herein, the term “biofilm” refers to an aggregate of microorganisms, including bacteria, archaea, viruses, protozoa, fungi or algae, in which cells are frequently embedded within a self-produced matrix of extracellular polymeric substance (EPS) and adhere to each other and/or to a surface.

As used herein, the term “cationized phytoglycogen” refers to phytoglycogen modified to include a positively charged functional group such as those containing a short chain quaternary ammonium compound. The short-chain quaternary ammonium compound includes at least one alkyl moiety having from 1 to 32 carbon atoms, preferably 1 to 30 carbon atoms, and more preferably 1 to 24 carbon atoms, unsubstituted or substituted with one or more N, 0, S, or halogen atoms. In a preferred embodiment, the short-chain quaternary ammonium compound includes at least one alkyl moiety having from 1 to 16 carbon atoms. In one embodiment, the modifier is 3-(trimethylammonio)2-hydroxypropy-1-yl with a degree of substitution of 0.05 to 2.0, preferably 0.3 to 1.2.

As used herein, the term “extracellular polymeric substance” (EPS) refers to self-produced matrix by a microorganism, and any incorporated extraneous materials, generally composed of extracellular biopolymers in various structural forms including, for example, extracellular DNA, proteins, lipids and polysaccharides.

As used herein, “therapeutically effective amount” refers to an amount effective, at dosages and for a particular period of time necessary, to achieve the desired therapeutic result. A therapeutically effective amount of the pharmacological agent may vary according to factors such as the disease state, age, sex, and weight of the individual, and the ability of the pharmacological agent to elicit a desired response in the individual. A therapeutically effective amount is also one in which any toxic or detrimental effects of a pharmacological agent are outweighed by the therapeutically beneficial effects.

As used herein “patient” refers to an animal being treated for an infection, which in one embodiment may be a vertebrate, in one embodiment a mammal, in one embodiment, a human patient. As used herein, the term “treatment” refers to administering a composition of the invention to effect an alteration or improvement of the disease or condition, which may include alleviating one or more symptom thereof. The use may be prophylactic. Prevention, amelioration, and/or treatment may require administration of multiple doses at regular intervals, or prior to onset of the disease or condition to alter the course of the disease or condition.

The present disclosure relates to anti-infective compositions comprising glycogen or phytoglycogen nanoparticles, including modified glycogen or phytoglycogen such as cationized phytoglycogen functionalized with short chain quaternary ammonium compounds (“phytoglycogen nanoparticle(s)”). Further, the present disclosure relates to compositions comprising phytoglycogen nanoparticles for use as anti-infectives. In one embodiment, the anti-infectives are used as a biofilm inhibitor.

In one embodiment, the nanoparticles may be used as a component of an antibiotic treatment to reduce the amount of antibiotic required to achieve the desired therapeutic result.

Phytoglycogen is composed of molecules of α-D glucose chains having an average chain length of 11-12, with 1→4 linkage and branching point occurring at 1→6 and with a branching degree of about 6% to about 13%. In one embodiment, phytoglycogen includes both phytoglycogen derived from natural sources and synthetic phytoglycogen. As used herein the term “synthetic phytoglycogen” includes glycogen-like products prepared using enzymatic processes on substrates that include plant-derived material e.g. starch.

The yields of most known methods for obtaining glycogen and phytoglycogen and most commercial sources of glycogen and phytoglycogen are highly polydisperse products that include both glycogen or phytoglycogen particles, as well as other products and degradation products of glycogen or phytoglycogen, which will render them less effective in the compositions and methods described herein. Accordingly, suitably substantially monodisperse glycogen or phytoglycogen is used. These substantially monodisperse glycogen or phytoglycogen nanoparticles have a low polydispersity index. In a preferred embodiment, monodisperse phytoglycogen nanoparticles are used. In one embodiment, the monodisperse phytoglycogen nanoparticles are PhytoSpherix™ by Mirexus Biotechnologies, Inc.

In one embodiment, phytoglycogen refers to monodisperse phytoglycogen nanoparticles manufactured according to methods described herein. The described methods enable production of substantially spherical nanoparticles, which are a single phytoglycogen molecule.

In a preferred embodiment, monodisperse cationized phytoglycogen nanoparticles are used.

Detailed below are monodisperse compositions of phytoglycogen nanoparticles. The monodisperse and particulate nature of the compositions described herein are associated with properties that render them highly suitable for use in anti-infective applications. Further, these phytoglycogen nanoparticles suitably have a size of between about 30 and 150 nm, in one embodiment, between 60 and 110 nm.

Accordingly, in a preferred embodiment, anti-infective compositions of monodisperse phytoglycogen nanoparticles are used.

Phytoglycogen nanoparticles as taught herein have a number of properties that make them particularly suitable for use in anti-infective compositions. Many existing drugs are rapidly eliminated from the body leading to a need for increased dosages. The compact spherical nature of phytoglycogen nanoparticles is associated with efficient cell uptake, while the highly-branched nature and high molecular weight of phytoglycogen is believed to be associated with slow enzymatic degradation and increased intravascular retention time, respectively.

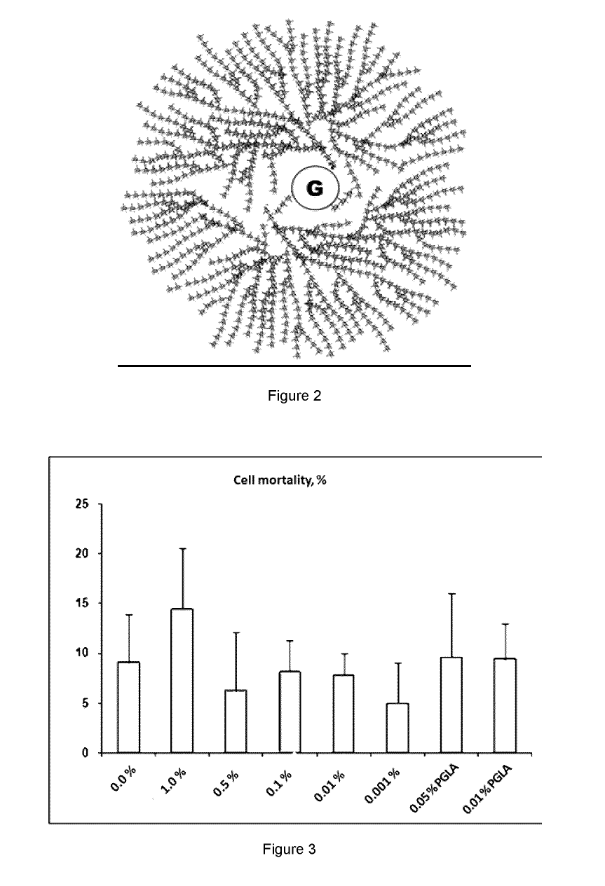

As shown in FIG. 2, each phytoglycogen particle is a single molecule, made of highly branched glucose homopolymer characterized by very high molecular weight (up to 10⁷ Da). This homopolymer consists of α-D-glucose chains with 1-4 linkage and branching points occurring at 1→6 and with branching degree about 10%. These particles are spherical and can be manufactured with different sizes, in the range of 30 to 150 nm in diameter by varying the starting material and filtering steps. The high density of surface groups on the phytoglycogen nanoparticles results in a variety of unique properties of phytoglycogen nanoparticles, such as fast dissolution in water, low viscosity and shear thinning effects for aqueous solutions at high concentrations of phytoglycogen nanoparticles. This is in contrast to high viscosity and poor solubility of linear and low-branched polysaccharides of comparable molecular weight. Furthermore, it allows formulation of highly concentrated (up to 30%) stable dispersions in water or DMSO.

As demonstrated by the Examples, the present inventors have found that phytoglycogen nanoparticles can be accumulated intracellularly by different types of cells.

When phytoglycogen nanoparticles are internalized, the nanoparticles are digested by cellular hydrolases. The rate of breakdown can be controlled by the degree of phytoglycogen derivatization by small molecules, e.g., methylation, hydroxypropylation, (which affect the affinity of hydrolases to polysaccharide chain and, therefore, the rate of hydrolysis).

The phytoglycogen nanoparticles can be further modified with specific tissue targeting molecules.

The phytoglycogen nanoparticles are non-toxic, have no known allergenicity, and can be degraded by glycogenolytic enzymes (e.g. amylases and phosphorylases) of the human body. The products of enzymatic degradation are non-toxic molecules of glucose.

Phytoglycogen nanoparticles are generally photostable and stable over a wide range of pH, electrolytes, e.g. salt concentrations.

United States patent application publication no. United States 2010/0272639 A1, assigned to the owner of the present invention and the disclosure of which is incorporated by reference in its entirety, provides a process for the production of glycogen nanoparticles from bacterial and shell fish biomass. The processes disclosed generally include the steps of mechanical cell disintegration, or by chemical treatment; separation of insoluble cell components by centrifugation; elimination of proteins and nucleic acids from cell lysate by enzymatic treatment followed by dialysis which produces an extract containing crude polysaccharides, lipids, and lipopolysaccharides (LPS) or, alternatively, phenol-water extraction; elimination of LPS by weak acid hydrolysis, or by treatment with salts of multivalent cations, which results in the precipitation of insoluble LPS products; and purification of the glycogen enriched fraction by ultrafiltration and/or size exclusion chromatography; and precipitation of glycogen with a suitable organic solvent or a concentrated glycogen solution can be obtained by ultrafiltration or by ultracentrifugation; and freeze drying to produce a powder of glycogen. Glycogen nanoparticles produced from bacterial biomass were characterized by Mwt 5.3-12.7×10⁶ Da, had particle size 35-40 nm in diameter and were monodisperse.

Methods of manufacturing monodisperse compositions of phytoglycogen are disclosed in the International patent application entitled “Phytoglycogen Nanoparticles and Methods of Manufacture Thereof”, published under the international application publication no WO2014/172786 and the disclosure of which is incorporated by reference in its entirety. In one embodiment, the described methods of producing monodisperse phytoglycogen nanoparticles include: a. immersing disintegrated phytoglycogen-containing plant material in water at a temperature between about 0 and about 50° C.; b. subjecting the product of step (a.) to a solid-liquid separation to obtain an aqueous extract; c. passing the aqueous extract of step (b.) through a microfiltration material having a maximum average pore size of between about 0.05 μm and about 0.15 μm; and d. subjecting the filtrate from step c. to ultrafiltration to remove impurities having a molecular weight of less than about 300 kDa, in one embodiment, less than about 500 kDa, to obtain an aqueous composition comprising monodisperse phytoglycogen nanoparticles. In one embodiment of the method, the phytoglycogen-containing plant material is a cereal selected from corn, rice, barley, sorghum or a mixture thereof. In one embodiment, step c. comprises passing the aqueous extract of step (b.) through (c.1) a first microfiltration material having a maximum average pore size between about 10 μm and about 40 μm; (c.2) a second microfiltration material having a maximum average pore size between about 0.5 μm and about 2.0 μm, and (c.3) a third microfiltration material having a maximum average pore size between about 0.05 and 0.15 μm. The method can further include a step (e.) of subjecting the aqueous composition comprising monodisperse phytoglycogen nanoparticles to enzymatic treatment using amylosucrose, glycosyltransferase, branching enzymes or any combination thereof. The method avoids the use of chemical, enzymatic or thermal treatments that degrade the phytoglycogen material. The aqueous composition can further be dried.

In one embodiment, the nanoparticles are produced from sweet corn starting material (Zea mays var. saccharata and Zea mays var. rugosa). In one embodiment, the sweet corn is of standard (su) type or sugary enhanced (se) type. In one embodiment, the composition is produced from dent stage or milk stage kernels of sweet corn. Unlike glycogen from animal or bacterial sources, use of phytoglycogen reduces the risk of contamination with prions or endotoxins, which may be associated with these other sources.

The polydispersity index (PDI) of a composition of nanoparticles can be determined by the dynamic light scattering (DLS) technique and, in this embodiment, PDI is determined as the square of the ratio of standard deviation to mean diameter (PDI=(a/d)². PDI can also be expressed through the distribution of the molecular weight of polymer and, in this embodiment, is defined as the ratio of Mw to Mn, where Mw is the weight-average molar mass and Mn is the number-average molar mass (hereafter this PDI measurement is referred to as PDI*). In the first case, a monodisperse material would have a PDI of zero (0.0) and in the second case the PDI* would be 1.0.

In one embodiment, there is provided an anti-infective composition that comprises, consists essentially of, or consists of a composition of monodisperse phytoglycogen nanoparticles. Suitably, these nanoparticles are modified as described further below. In one embodiment, the anti-infective composition comprises, consists essentially of, or consists of a composition of monodisperse phytoglycogen nanoparticles having a PDI of less than about 0.3, less than about 0.2, less than about 0.15, less than about 0.10, or less than 0.05 as measured by dynamic light scattering. In one embodiment, the anti-infective composition comprises, consists essentially of, or consists of a composition of monodisperse phytoglycogen nanoparticles having a PDI* of less than about 1.3, less than about 1.2, less than about 1.15, less than about 1.10, or less than 1.05 as measured by SEC MALS.

In one embodiment, the anti-infective composition comprises, consists essentially of, or consists of a composition of monodisperse phytoglycogen nanoparticles having an average particle diameter of between about 30 nm and about 150 nm. In one embodiment, the anti-infective composition comprises, consists essentially of, or consists of a composition of monodisperse phytoglycogen nanoparticles having an average particle diameter of about 60 nm to about 110 nm. In other embodiments, there is provided compositions comprising, consisting essentially of, or consisting of, nanoparticles having an average particle diameter of about 40 to about 140 nm, about 50 nm to about 130 nm, about 60 nm to about 120 nm, about 70 nm to about 110 nm, about 80 nm to about 100 nm. These nanoparticles may be modified as described further below.

The methods of producing phytoglycogen nanoparticles as detailed in Example 1 and in the international patent application no. PCT/CA2014/000380, published under the international application publication no WO/2014/172786, entitled “Phytoglycogen Nanoparticles and Methods of Manufacture Thereof”, are amenable to preparation under pharmaceutical grade conditions.

### Chemical Modification of Phytoglycogen Nanoparticles

To impart specific properties to phytoglycogen nanoparticles, they can be chemically modified via numerous methods common for carbohydrate chemistry.

Accordingly, in a preferred embodiment, the phytoglycogen nanoparticles are modified. The resulting products are referred to herein interchangeably as functionalized nanoparticles or derivatives. Functionalization can be carried out on the surface of the nanoparticle, or on both the surface and the interior of the particle, but the structure of the glycogen or phytoglycogen molecule as a single branched homopolymer is maintained. In one embodiment, the functionalization is carried out on the surface of the nanoparticle. As will be understood by those of skill in the art, chemical modifications should be non-toxic and generally safe for human consumption. The chemical character of phytoglycogen nanoparticles produced according to methods described above may be changed from their hydrophilic, slightly negatively charged native state to be positively and/or negatively charged, or to be partially or highly hydrophobic. Chemical processing of polysaccharides is well known in the art. See for example J. F Robyt, Essentials of Carbohydrate Chemistry, Springer, 1998; and M. Smith, and J. March, March's Advanced Organic Chemistry: Reactions, Mechanisms, and Structure Advanced Organic Chemistry, Wiley, 2007.

As will be described further below, nanoparticles modified to have a positive charge demonstrate anti-infective activity, including antimicrobial activity.

The nanoparticles can be functionalized either directly or indirectly, where one or more intermediate linkers or spacers can be used. The nanoparticles can be subjected to one or more than one functionalization steps including two or more, three or more, or four or more functionalization steps.

Various derivatives can be produced by chemical functionalization of hydroxyl groups of phytoglycogen, either by etherification with a suitably functionalized alkyl group, by interconversion of the hydroxyl group into another functional group, or by oxidation. Such functional groups include, but are not limited to, nucleophilic and electrophilic groups, and acidic and basic groups, e.g., carbonyl groups, amine groups, thiol groups, carboxyl groups and their derivatives such as amide or esters, azide, nitrile, halogenide and pseudo-halogenide such as tosyl, mesyl or triflate, and hydrocarbyl groups such as alkyl, vinyl, phenyl, benzyl, propargyl and allyl groups. Amino groups can be primary, secondary, tertiary, or quaternary amino groups, preferably quaternary amino groups.

Functionalized nanoparticles can be further conjugated with various desired molecules, which are of interest for a variety of applications, such as biomolecules, small molecules, therapeutic agents, micro- and nanoparticles, pharmaceutically active moieties, macromolecules, diagnostic labels, chelating agents, dispersants, charge modifying agents, viscosity modifying agents, surfactants, coagulation agents and flocculants, as well as various combinations of these chemical compounds. In certain embodiments, two or more different chemical compounds are used to produce multifunctional derivatives.

In one embodiment, the functionalized nanoparticles are modified with a quaternary ammonium compound.

The reactivity of hydroxyl groups on glucose subunits is low. Even so, reactions are possible at high pH with epoxides, alkyl halides or anhydrides, forming the corresponding ether or ester linkages. Water-soluble chemicals with epoxide or anhydride functionalities react at basic pH (8-13) with phytoglycogen nanoparticles (in the presence of an appropriate catalyst). Although derivatization in aqueous environment is often preferable, some reactions (e.g. with alkyl halides) are best conducted in organic solvents such as dimethyl sulfoxide (DMSO), dimethyl formamide (DMF), dimethyl acetamide or pyridine, or mixtures of the aforementioned with salts such as lithium chloride or tetrabutylammonium fluoride. As will be apparent to one of skill in the art, water-soluble compounds with low toxicity and reactive at relatively mild conditions are particularly suitable.

A simple approach to increasing the reactivity of hydroxyl groups is the selective oxidation of glucose hydroxyl groups at positions of C-2, C-3, C-4 and/or C-6, yielding carbonyl or carboxyl groups or carboxyl. There is a wide spectrum of redox initiators which can be employed, such as persulfate, periodate (e.g. potassium periodate, bromine, sodium chlorite (2,2,6,6-tetramethylpiperidin-1yl)oxidanyl, commonly known as TEMPO, and Dess-Martin periodinane.

Phytoglycogen nanoparticles functionalized with carbonyl groups are readily reactive towards compounds bearing primary or secondary amine groups. This results in imine formation (eq. 1) which can be further reduced to amines with a reducing agent e.g., sodium borohydride (eq. 2). This reduction step provides an amino-product which is more stable than the imine intermediate, and also converts unreacted carbonyls in hydroxyl groups. The elimination of carbonyls significantly reduces the possibility of non-specific interactions of derivatized nanoparticles with non-targeting molecules (e.g. plasma proteins).

P/G NANO—CH═O+H₂N—R→P/G NANO—CH═NH—R+H₂O (eq. 1)

reducing agent

P/G NANO—CH═NH—R→P/G NANO—CH₂—NH—R (eq. 2)

Carboxyl groups can be activated using coupling reagents such as N,N′-Dicyclohexylcarbodiimide (DCC), 1-Ethyl-3-(3-dimethylaminopropyl)carbodiimide (EDC), or 1,1′-Carbonyldiimidazole (CDI), with or without the addition of auxiliary reagents such as 1-Hydroxybenzotriazol (HOBt) or N-Hydroxysuccinimide (NHS). The activated carboxylate then reacts under very mild conditions with nucleophiles such as amino or hydroxy groups (examples 9, 10). This type of activation can either be used to activate carboxyl groups on a small molecule, and react it with hydroxy groups of native phytoglycogen or amino groups of aminated phytoglycogen; or it can be used to activate carboxyl groups on oxidized phytoglycogen and attach an amino-containing small molecule to it (example 12).

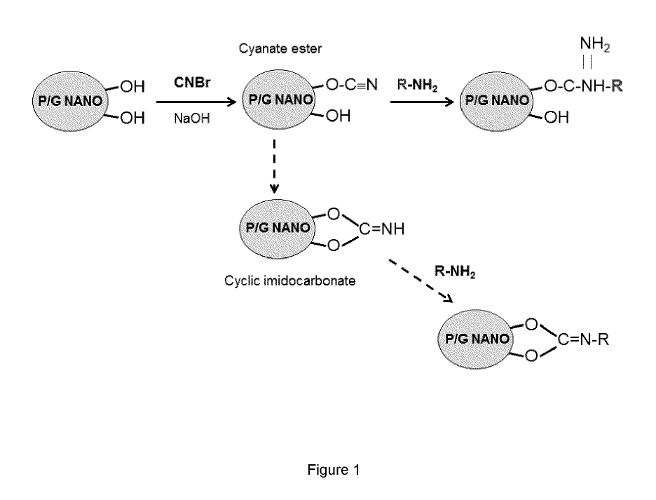

In certain embodiments, the nanoparticles described are functionalized via a process of cyanylation. This process results in the formation of cyanate esters and imidocarbonates on polysaccharide hydroxyls. These groups react readily with primary amines under very mild conditions, forming covalent linkages (FIG. 1). Cyanylation agents such as cyanogen bromide and 1-cyano-4-diethylamino-pyridinium (CDAP) can be used for functionalization of the nanoparticles.

A chemical compound bearing a functional group capable of binding to the functional groups present on phytoglycogen or modified phytoglycogen can be directly attached to the nanoparticle. However, for some applications chemical compounds may be attached via a polymer spacer or a “linker”. These can be homo- or hetero-bifunctional linkers bearing functional groups such as amino, carbonyl, carboxyl, sulfhydryl, succimidyl, maleimidyl, isocyanate, (e.g. diaminohexane, ethylene glycobis(sulfosuccimidylsuccinate), disulfosuccimidyl tartarate, dithiobis(sulfosuccimidylpropionate), aminoethanethiol, etc.)

The antimicrobial activity of modified phytoglycogen nanoparticles functionalized with quaternary ammonium compounds may be further enhanced by modifying its hydrophobicity Therefore, in a preferred embodiment, the glycogen or phytoglycogen nanoparticle is double-modified with both quaternary ammonium and hydrophobic groups. The hydrophobic interactions can be fine-tuned by choosing an appropriate degree of substitution and hydrophobic functional group. Example functional groups include, but are not limited to aliphatic alkyl, alkenyl, alkynyl or benzyl ethers and esters or trialkylsilyl ethers of chain lengths between 1 and 24 (Examples 5-7).

In one embodiment, there is provided a method of treating a subject suffering from a microbial infection comprising administering to the subject a therapeutically effective amount of a composition as described herein. In one embodiment, the composition comprises functionalized phytoglycogen nanoparticles having a positive surface charge. In one embodiment, the phytoglycogen nanoparticles are functionalized with a secondary, tertiary or quaternary ammonium group. In one embodiment, the composition comprises phytoglycogen nanoparticles functionalized with an amphiphilic group. In one embodiment, the composition comprises glycogen or phytoglycogen nanoparticles functionalized with quaternary ammonium compounds. In certain embodiments, phytoglycogen nanoparticles as described above may be functionalized and used without further conjugation.

In other embodiments, the nanoparticles may further be conjugated to other chemical compounds that can include biomolecules, small molecules, therapeutic agents, pharmaceutically active moieties, macromolecules, diagnostic labels, chelating agents, dispersants, surfactants, charge modifying agents, viscosity modifying agents, coagulation agents and flocculants, to name a few, as well as various combinations of the above.

Biomolecules which can be conjugated include peptides, enzymes, receptors, neurotransmitters, hormones, cytokines, cell response chemical compounds such as growth factors and chemotactic factors, antibodies, vaccines, haptens, toxins, interferons, ribozymes, anti-sense agents, and nucleic acids.

Anti-infective compositions according to one embodiment include functionalized monodisperse phytoglycogen nanoparticles conjugated to other molecule(s). In various embodiments, the phytoglycogen nanoparticles are further conjugated to a pharmaceutical. In various embodiments, the nanoparticles are conjugated to one or more of an antibiotic, an antifungal, an anti-parasite and/or anti-protozoal compound. Pharmaceutically useful moieties used as modifiers include hydrophobicity modifiers, pharmacokinetic modifiers, and biologically active modifiers.

Chemical compounds which are conjugated to phytoglycogen nanoparticles may have light absorbing, light emitting, fluorescent, luminescent, Raman scattering, fluorescence resonant energy transfer, and electroluminescence properties.

Two or more different chemical compounds can be used to produce multifunctional derivatives. For example, one chemical compound can be selected from the list of specific binding biomolecules, such as antibody and aptamers, while the second compound would be selected from the list of anti-infectives. For example, one chemical compound may be a cationic species, while the second compound may be an antibiotic.

Loading efficiency depends on the molecular weight and properties (charge, hydrophobicity, etc.) of the molecules to be conjugated. Degree of substitution is expressed as % of anhydroglucose units derivatized with the drug. E.g. if the drug has a molecular weight of 100 Da, and the degree of substitution is 50%, then 1 g of phytoglycogen nanoparticles would carry 0.31 g of the drug. For small molecules (<100 Da) a degree of substitution >30% was generally achieved, going as high as 100% for methyl groups. Larger molecules (which cannot penetrate the pore structure of the particles) can be conjugated only at the surface of the phytoglycogen nanoparticles, and the degree of substitution is lower, generally 0.1-2.0%.

### Anti-Infective Activity

As detailed in the Examples, the present inventors have developed compositions of phytoglycogen nanoparticles including functionalized forms thereof with properties that render them highly suitable for use in anti-infective applications.

In one embodiment, there is provided an anti-infective composition comprising, consisting of or consisting essentially of positively charged phytoglycogen nanoparticles. The surface of phytoglycogen nanoparticles can be made cationic through a number of techniques, as described above.

In one embodiment, there is described an anti-infective composition comprising phytoglycogen, preferably positively charged nanoparticles of phytoglycogen. In one embodiment, the composition further comprises a carrier, which in one embodiment is a pharmaceutically acceptable carrier.

In one embodiment, the nanoparticles are modified with an amphiphilic compound.

Cationic modifications to phytoglycogen nanoparticles, which can render them useful as anti-infectives may include secondary, tertiary or quaternary amino groups and, in particular, modifications with quaternary-ammonium derivatives. In one embodiment, the quaternary ammonium derivatives can be selected from hydroxypropyl-trimethylammonium and hydroxypropyl-alkyl-dimethylammonium, wherein alkyl is a aliphatic C₂ to C₃₂ aliphatic hydrocarbon, such as, but not limited to lauryl-, myristyl- or stearyl-. In another embodiment, the alkyl is a C₂ to C₃₂ hydrocarbon, preferably C₂ to C₃₀, more preferably C₂ to C₂₄.

The surfaces of bacteria are typically anionic, and without wishing to be bound by a theory, the inventors hypothesize that the creation of localized high densities of cationized groups on the surface of a phytoglycogen nanoparticle create a cumulative charge-based effect capable of affecting bacterial growth and physiology.

As demonstrated by the Examples, anti-infective activity against Escherichia coli, Bacillus subtilis, Pseudomonas aeruginosa and Candida utilis is shown in the presence of a composition of phytoglycogen nanoparticles.

In a preferred embodiment, the phytoglycogen nanoparticles are modified to a cationized form functionalized with short chain quaternary ammonium compounds.

As shown in the Examples, increased anti-infective susceptibility to a variety of classes of antibiotics is shown against P. aeruginosa, E. coli, B. subtilis and C. utilis following incubation in the presence of cationized phytoglycogen.

In one embodiment, phytoglycogen nanoparticles are co-administered with an antibiotic, which may be selected from but is not limited to Penicillins, Carboxypenicillins, Aminopenicillins, Glycopeptides, Quinolones, Cephalosporins, Macrolides, Fluoroquinolones, Phenicols, Sulfonamides, Tetracyclines, Aminocoumarins, Lipopeptides or Aminoglycosides.

In one embodiment, phytoglycogen nanoparticles are co-administered with an antifungal, which may be selected from but is not limited to a Polyene, Imidazole, Triazole, Thiazole, Allylamine, or Echinocandin antifungal.

In one embodiment, there is provided an anti-infective composition comprising both phytoglycogen nanoparticles and an antibiotic, which may be selected from but is not limited to Penicillins, Carboxypenicillins, Aminopenicillins, Glycopeptides, Quinolones, Cephalosporins, Macrolides, Fluoroquinolones, Phenicols, Sulfonamides, Tetracyclines, Aminocoumarins, Lipopeptides, cationic antimicrobial peptides, or Aminoglycosides.

In one embodiment, there is provided an anti-infective composition comprising both phytoglycogen nanoparticles and an antifungal, which may be selected from but is not limited to a Polyene, Imidazole, Triazole, Thiazole, Allylamine, or Echinocandin antifungal.

In one embodiment, the nanoparticles are conjugated to an antibiotic, which may be selected from but is not limited to Penicillins, Carboxypenicillins, Aminopenicillins, Glycopeptides, Quinolones, Cephalosporins, Macrolides, Fluoroquinolones, Phenicols, Sulfonamides, Tetracyclines, Aminocoumarins, Lipopeptides, Cationic antimicrobial peptides, or Aminoglycosides.

In one embodiment, the nanoparticles are conjugated to an antifungal, which may be selected from but is not limited to a Polyene, Imidazole, Triazole, Thiazole, Allylamine, or Echinocandin antifungal.

In one embodiment, the anti-infective composition further comprises a pharmaceutically acceptable carrier or excipient.

Anti-infective compositions as described herein may be used to treat bacterial, fungal or parasitic infections and may also be used prophylactically.

Also provided is a method of treating a microbial infection comprising administering a therapeutically effective amount of an anti-infective composition as described herein to a subject in need thereof. In one embodiment, the microbial infection is a fungal infection. In one embodiment, the microbial infection is a bacterial infection.

In one embodiment, phytoglycogen nanoparticles are used as a co-therapeutic not as an antibiotic, but as an anti-infective to regulate virulence and pathogenicity of microorganisms.

The infection may be an intracellular infection.

The anti-infective activity of phytoglycogen nanoparticles may operate in whole or in part by decreasing or inhibiting biofilm formation, maintenance or growth as discussed more particularly below.

In some embodiment, the anti-infective activity of phytoglycogen nanoparticles may operate through the attenuation or modification of the production of virulence factors by an infective agent such as a bacterium, yeast, fungus, or parasite, resulting in a diminished ability to cause infection.

In various embodiments, the infection is in the liver, upper and lower respiratory tracts (e.g.

sinusitis, whooping cough, pneumonia), eyes, ears, gum and/or mouth (e.g. periodontitis and gingivitis), kidney, intestinal tract, genito-urinary tract and bladder, blood (e.g., bacteraemia), brain, meninges, spinal cord, bone, gut and/or cardiac system. In another embodiment, the infection is a wound or skin infection.

In one embodiment, there is provided a method of treating an intracellular infection comprising administering a therapeutically effective amount of a composition as described herein to a subject in need thereof. In various embodiments, the intracellular infection may be caused by microorganisms, including, but not limited to, Legionella pneumophila, Candida spp., Salmonella spp., invasive E. coli spp. Listeria monocytogenes, Rickettsia rickettsii, Chlamydia, Shigella spp., Francisella tularensis, Yersinia pestis, Neisseria, Brucella spp., Bartonella spp., Staphylococcus aureus, Coxiella bumettii, Cryptococcus neoformans, Histoplasmata capsulatum, and/or Pneuomcystis jirovecii/carinii.

Infections of the upper and/or lower respiratory tract and/or airways may be bacterial or fungal in nature. Common causes of bacterial lung infections include Streptococcus pneumoniae, Haemophilus species, Klebsiella pneumoniae, Staphylococcus aureus, Mycobacterium tuberculosis, and Pseudomonas aeruginosa. Common pathogens causing fungal lung infections include Histoplasma capsulatum, Coccidioides immitis, Blastomyces dermatitidis, Paracoccidioides brasiliensis, Pneumocytis jirovecii/carinii, Candida spp., Aspergillus spp., Mucor spp. and Cryptococcus neoformans.

In one embodiment, there is provide a method of treating an intracellular infection within the lungs comprising administering a therapeutically effective amount of a composition as described herein to a subject in need thereof.

In one embodiment, the anti-infective may act to decrease or inhibit biofilm formation, maintenance or growth.

Administration to the lung may be by, although is not limited to, inhalation.

In one embodiment, phytoglycogen nanoparticles are used as a co-therapeutic or as part of a conjugated anti-infective for the treatment of a pulmonary infection.

In one embodiment, phytoglycogen nanoparticles can be used as a co-therapeutic for the treatment of chronic pulmonary infections of P. aeruginosa, which are typical of individuals with cystic fibrosis.

Gastroenteritis may be caused by a number of microorganisms, including, but not limited to, Yersinia enterocolitica, Clostridium perfringens, Clostridium difficile, Helicobacter pylori, Staphylococcus aureus, Shigella spp., Pseudomonas aeruginosa, Salmonella spp., Campylobacter jejuni, Escherichia coli, Candida spp. In one embodiment, compositions as described herein can be used to treat gastroenteritis.

In one embodiment, the anti-infective may act to decrease or inhibit biofilm formation, maintenance or growth in the intestines.

Administration to the intestines may be by, although is not limited to, orally or by suppository.

In one embodiment, the infection is a skin infection and the composition is topically applied. Bacterial skin infections include, but are not limited to acne, impetigo, cellulitis and streptococcal infections. Fungal skin infections include but are not limited to Tinea pedis (athlete's foot), Tinea cruris (jock itch), Tinea corporis (ringworm) and yeast infections.

In various embodiments, the infection may be associated with a cut, blister, burn, insect bite, surgical wound, injection site or catheter insertion site.

In one embodiment, the infection is of the hair or nails. In one embodiment, there is provided an anti-infective shampoo comprising compositions as described herein.

As described further below, the compositions as described herein can be suitably formulated as powders, lotions, gels, foams, sprays or ointments.

Uses also include antibacterial skin sanitizers, and surface sanitizers. In one embodiment phytoglycogen nanoparticles may be conjugated with an active compound of an antiseptic or sanitizer. The use of phytoglycogen nanoparticles as a surface sanitizer may operate through inhibition of cell growth or cell death, inhibition of biofilm formation, biofilm dissolution or disruption of quorum sensing as discussed more particularly below.

In another embodiment, anti-infective compositions as described herein may be used as anti-infective coatings for medical devices, such as diagnostic devices, implanted devices such as pacemakers, artificial joints, stents, and catheters. In other embodiments, the compositions may be impregnated into or coated onto bandages, surgical suture thread, wound dressings, wipes, towelettes, patches or sponges, or incorporated into bone cement. In one embodiment, the infections are associated with implanted devices such as indwelling catheters, pacemakers, artificial joints, auditory implants, and stents.

In one embodiment, anti-infective compositions of the present invention are used in the treatment of intracellular infections. Many pathogenic bacteria can infect and survive within host cells, including cells of the immune system (monocytes/phagocytes) that are supposed to kill them. It is more challenging to treat such infections since once within the cell interior the pathogens are somewhat protected from antibiotics. Many antibiotics show a lack of accumulation whether in phagocytic or non-phagocytic cells and tissues in general due to low cell membrane permeability, fast efflux etc. Often, higher antibiotics doses are needed to effectively kill bacteria in the cell interior. The phytoglycogen nanoparticles as described herein provide a solution to this problem by providing targeted delivery of antibiotics to host cells, e.g., macrophages, and to reach effective concentration to kill intracellular bacteria. As demonstrated by the Examples, phytoglycogen nanoparticles can carry compounds across the cell membrane and were shown to accumulate within the cytoplasm.

Phytoglycogen nanoparticles as described herein can stabilize peptides e.g. antimicrobial peptides. Protein and peptides stored in solution or frozen or formulated in dry formulations (e.g. spray dried or freeze-dried) tend to lose their efficacy over time due to aggregation, decomposition, denaturation, oxidation and deamidation. While the stabilizing activity can help improve shelf life, it may also allow for less onerous storage requirements e.g. limiting the requirement for refrigeration. Phytoglycogen nanoparticles can stabilize organic compounds. As mentioned above, the highly-branched nature of glycogen and phytoglycogen is associated with slow enzymatic degradation. Without wishing to be bound by a theory, the monodisperse phytoglycogen nanoparticles as described herein can provide both structural stabilization to protein and peptide solutions and inhibit degradation through steric hindrance of enzymatic degradation.

As demonstrated by the Examples, the conjugated antibiotic-phytoglycogen may act without being cleaved; equally, it may act as a cleaved product.

A biofilm is a sessile community of microorganisms in which the cells are adhered to one another and also often to a surface. These adherent cells are physiologically distinct from planktonic microbial cells which are single cells that are suspended in a liquid medium. The adherent cells found in a biofilm are embedded within a self-produced matrix of extracellular polymeric substance (EPS); the EPS may also comprise incorporated extraneous materials. This EPS is a conglomeration generally composed of extracellular biopolymers in various structural forms. The EPS allows the microorganisms living in this type of environment to be less susceptible to anti-infectives in some cases. The EPS confers benefits to microorganisms including, but not limited to, enabling 3-D architecture, cellular organization, creation of micro-environments, and the generation of a plethora of phenotypes. Collectively these enable key features of biofilm communities, including decreased susceptibility to anti-infectives and other inimical agents, reduced predation and invasion, evasion of components of the immune response and the consequent difficulty to eradicate infections.

Biofilms are present in the natural environment, and are common in hospitals and industrial settings. Biofilms can form on living and non-living surfaces, including native tissues and medical devices. In cases where microorganisms succeed in forming a biofilm on or within a host, including human hosts, chronic and untreatable infection can result.

As detailed in the Examples, the present inventors have developed compositions of phytoglycogen nanoparticles including functionalized forms thereof with properties that render them highly suitable for use to decrease or inhibit biofilm formation, maintenance and growth.

Without wishing to be bound by a theory, the present inventors hypothesize that treatment of biofilms with functionalized phytoglycogen nanoparticles may decrease or inhibit biofilm formation, maintenance and growth through charge-based mechanisms which result in disruption of and/or reduction in biofilm formation and/or enhanced biofilm dissolution.

Further, as discussed below, charge-based interactions may interfere with quorum sensing-related processes, leading to the attenuation of the production of virulence factors.

The modification or attenuation of the production of virulence factors may alter a cellular phenotype that modulates cell-extracellular interactions, or that decreases or inhibits the production of toxins, biofilms or enzymes.

Quorum sensing is a density dependent cell-to-cell signaling system that regulates a range of bacterial processes. It is a two-step process that involves the production and release of signals by the bacteria into the environment and signal detection by a receptor (sensing). When a threshold concentration is reached, indicating a quorum, this directs up- or down-regulation of genes thereby enabling co-ordinated responses of single cells and concerted population responses.

Quorum sensing is pivotal for a number of bacterial processes including infection, production of virulence factors, colonisation of surfaces and biofilm formation. Since quorum sensing is established as a central factor in the progression of infectious disease by microorganisms, there has been a drive to develop strategies which interfere with quorum sensing, thereby attenuating virulence.

In addition to the role of quorum sensing in regulating production of virulence factors and phenotypes consistent with virulence and pathogenesis, quorum sensing signals may also interface with the host. Certain quorum sensing signals produced have immunomodulatory properties which alter the response of the host immune system and coordinate subversion of host defenses.

Without wishing to be bound by a theory, the present inventors hypothesize that phytoglycogen nanoparticles may interfere with quorum sensing processes to regulate the production of virulence factors and interface with the host to alter the response of the host immune system.

As demonstrated in the Examples, down-regulation and disruption of quorum sensing regulated processes in P. aeruginosa occurs in the presence of cationized phytoglycogen. Further, as demonstrated in the Examples, reduced pyocyanin production, decreased biofilm formation, enhanced biofilm dissolution and decreased biofilm accretion occurs in the presence of cationized phytoglycogen.

In one embodiment, phytoglycogen nanoparticles are used as a skin or surface sanitizer as described above. In one embodiment, the phytoglycogen nanoparticles can be used as a gel or in a semi-solid state as described above.

In one embodiment, phytoglycogen nanoparticles can be used in a spray, optionally an aerosol form. In one embodiment, the composition is a spray on product that can be used topically on a human or on a non-living surface. In another embodiment, phytoglycogen nanoparticles can be inhaled in an aerosolized form.

In another embodiment, phytoglycogen nanoparticles can be used internally to decrease or inhibit biofilm formation, maintenance or growth.

As shown in the Examples, decreased motility and pyocyanin production of P. aeruginosa is observed in the presence of cationized phytoglycogen. Swarming motility and pyocyanin production are two virulence factors regulated by quorum sensing processes in P. aeruginosa. Cationized phytoglycogen may act as a co-therapeutic in the management of chronic P. aeruginosa infections typical within the respiratory tracts of patients with cystic fibrosis, not as an antibiotic per se but as an anti-infective to regulate virulence and pathogenicity.

In one embodiment, phytoglycogen nanoparticles are used in conjunction with an antibiotic, an antifungal, or an antiparasitic as described above. In another embodiment, phytoglycogen nanoparticles are conjugated to one or more of an antibiotic, an antifungal agent, an anti-parasite and/or anti-protozoal compound, an anti-adhesion molecule, an analgesic, an anticoagulant, a local anesthetic, and an imaging agent as described above.

In one embodiment, phytoglycogen nanoparticles are used as a co-therapeutic not as an antibiotic but as an anti-infective to regulate virulence and pathogenicity of microorganisms. In one embodiment, the microorganisms are in a planktonic population. In another embodiment, the microorganisms are in a biofilm community.

### Formulation and Administration

The nanoparticles of the invention may also be admixed, encapsulated, or otherwise associated with other molecules, molecule structures or mixtures of compounds and may be combined with any pharmaceutically acceptable carrier or excipient. As used herein, a “pharmaceutically carrier” or “excipient” can be a pharmaceutically acceptable solvent, suspending agent or any other pharmacologically inert vehicle for delivering functionalized phytoglycogen nanoparticles, whether alone or conjugated to a biologically active or diagnostically useful molecule, to an animal. The excipient may be liquid or solid and is selected, with the planned manner of administration in mind, so as to provide for the desired bulk, consistency, etc., when combined with phytoglycogen nanoparticles and the other components of a given pharmaceutical composition. Examples of pharmaceutically acceptable carriers include one or more of water, saline, phosphate buffered saline, glycerol, ethanol, propylene glycol, 1,3-butylene glycol, dimethyl sulfoxide, N,N-dimethylacetamide and the like, as well as combinations thereof. Pharmaceutically acceptable carriers may further comprise minor amounts of auxiliary substances such as wetting or emulsifying agents, preservatives or buffers, which enhance the shelf life or effectiveness of the pharmacological agent.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers, finely divided solid carriers, or both, and then, if necessary, shaping the product (e.g., into a specific particle size for delivery).

For the purposes of formulating pharmaceutical compositions, monodisperse phytoglycogen nanoparticles prepared as taught herein, may be provided in a dried particulate/powder form or may be dissolved e.g. in an aqueous solution.

In various embodiments, where a low viscosity is desired, the phytoglycogen nanoparticle component as described herein may suitably be used in the anti-infective compositions in a concentration of up to about 25% w/w, about 20% w/w, about 15% w/w, about 10% w/w, about 5% w/w, about 1% w/w and between about 0.05 and 0.5%.

In applications where a high viscosity is desirable, the phytoglycogen nanoparticle component may be used in formulations in concentrations above about 25% w/w. In applications where a gel or semi-solid is desirable, concentrations up to about 35% w/w can be used, or the phytoglycogen nanoparticle component can be used in a mixture with viscosity builders or gelling agents.

The composition may be a water-based formulation or an alcohol-based formulation. Suitable alcohols include ethyl alcohol, propyl alcohol, isopropyl alcohol, ethylene glycol, propylene glycol, butylene glycol, dipropylene glycol, ethoxydiglycol, or glycerol or a combination thereof.

The anti-infective compositions as described herein may be administered in a number of ways depending upon whether local or systemic treatment is desired and upon the area to be treated. Without limiting the generality of the foregoing, the route of administration may be topical, e.g. administration to the skin or by inhalation or in the form of ophthalmic or optic compositions; enteral, such as orally (including, although not limited to in the form of tablets, capsules or drops) or in the form of a suppository; or parenteral, including e.g. subcutaneous, intravenous, intra-arterial or intra-muscular; or in an inhaled form for delivery to the airways and/or to the lungs.

In one embodiment, the anti-infective composition is a topical formulation for application to the skin, for transdermal delivery. The monodisperse nanoparticles disclosed herein are particularly useful as film-forming agents. Because the nanoparticles are monodisperse, uniform close-packed films are possible. The compositions form stable films with low water activity. Accordingly, when chemically modified, they may be used to attach and carry bio-actives across the skin. In various embodiments, the topical formulation may be in the form of a gel, cream, foam, lotion, spray or ointment.

In another embodiment, the anti-infective compositions of the present invention are in the form of an implant. In one embodiment, the biomedical compositions as described herein are used to form biomedical articles. Suitably, these implants and biomedical articles may be biocompatible, meaning that they will have no significant adverse effects on cells, tissue or in vivo function. Suitably, these implants and biomedical articles may be bioresorbable or biodegradable (in whole or in part). Examples of biomedical articles that can be formed in whole or in part using compositions as described herein include, without being limited to: tissue engineering scaffolds and related devices, wound dressings and bandages, suture threads, coating for implantable wires, implanted devices such as catheters, stents, angioplasty balloons and other devices.

In one embodiment, the anti-infective compositions of the present invention are in the form of a coating or film. These coatings and films can be used e.g. for coating dosage forms, including pills. They can also suitably be used in topical application, including as protective films or in wound healing film dressing formulations. The phytoglycogen nanoparticles can be used in water dispersions or can be mixed with other film-forming polymers, plasticizers such as polyols, glycerol, sorbitol, propylene glycol, and polyethylene glycol, together with hydrophobic modifiers (e.g., lipids, stearopten and beeswax), binders e.g., polyvinylpyrrolidone, active pharmaceutical ingredients (APIs), and anti-infectives. In this regard, modified glycogen and phytoglycogen nanoparticles with ionizable groups e.g., carboxyl, amino or hydrophobic groups can provide better moisturization, adhesion to surfaces, API dispersion and anti-infective properties.

In the case of coatings for catheters, stents etc., phytoglycogen nanoparticle compositions as described herein can also provide lubrication.

In various embodiments, modified phytoglycogen nanoparticles can be used to encapsulate important materials (e.g. another API) to provide enhanced thermal, oxidative and UV stability, e.g., an API can be dispersed in a glycogen or phytoglycogen solution and spray dried (the encapsulation providing protection from thermal and/or oxidative degradation).

In one embodiment, a further API can be first encapsulated in phytoglycogen nanoparticles and then introduced to the formulation.

### EXAMPLES

### Example 1

### Extraction of Phytoglycogen from Sweet Corn Kernels

1 kg of frozen sweet corn kernels (75% moisture content) was mixed with 2 L of deionized water at 20° C. and was pulverized in a blender at 3000 rpm for 3 min. Mush was centrifuged at 12,000×g for 15 min at 4° C. The combined supernatant fraction was subjected to cross flow filtration (CFF) using a membrane filter with 0.1 μm pore size. The filtrate was further purified by a batch diafiltration using membrane with MWCO of 500 kDa and at RT and diavolume of 6, where the diavolume is the ratio of total milliQ water volume introduced to the operation during diafiltration to retentate volume.

The retentate fraction was mixed with 2.5 volumes of 95% ethanol and centrifuged at 8,000×g for 10 min at 4° C. The retentate was mixed with 2.5 volumes of 95% ethanol and centrifuged at 8,000×g for 10 min at 4° C. The pellet containing phytoglycogen was dried in an oven at 50° C. for 24 hrs and then milled to 45 mesh. The weight of the dried phytoglycogen was 97 g.

According to dynamic light scattering (DLS) measurements, the phytoglycogen nanoparticles produced had particle size diameter of 83.0 nm and a polydispersity index of 0.081.

### Example 2

### Synthesis of 3-(trimethylammonio)-2-hydroxyprop-1-ylderivatized phytoglycogen

225 g of phytoglycogen was dispersed in 1500 ml of 0.5 M NaOH solution in water. Then 346 ml of a 69% solution of 2,3-epoxypropyltrimethylammonium chloride in water was added to the mixture in the course of 5 h. The mixture was stirred for 24 h at room temperature before adjusting the pH to 7.0 with 6.2 M HCl. The product is precipitated by addition of 2 l of ethanol and stored over night at −20° C. The precipitate is collected, washed three times with ethanol, and oven-dried at 80° C. to dryness. The degree of substitution (DS) of the product was assessed using NMR spectroscopy and was found to be 0.73.

Preparations of sterile phytoglycogen and modified phytoglycogen were obtained using one of two methods.

Method 1—Filter Sterilization of a Solution (Not Exceeding 2% wt/vol)

Solutions were sterilised by syringe-driven filtration through a sterile 0.2 μm pore size filter and the filtrate collected in a sterile container. Dry weights of filtrates were then determined to account for any reduction in concentration.

Method 2—Gamma Irradiation of Dry Material

Pre-weighed samples of phytoglycogen and its derivatives were aliquoted into glass vials and irradiated to a dose of 6 kGy. Gamma-irradiated materials (20 mg/mL of trypticase soy broth) were incubated at either 25° C. or 37° C. No growth was shown after for 48 hrs at which point the materials were deemed sterile.

### Example 3

### Synthesis of 3-(trimethylammonio)-2-hydroxyprop-1-yl derivatized phytoglycogen, alternative procedure

1 g of phytoglycogen is mixed with aqueous sodium hydroxide solution (different examples, between 0.125-2.5 mmol of NaOH dissolved in 1-5 ml of water) and heated to 45° C. In the course of 30 min, 3.07 ml of a 69% solution of 2,3-epoxypropyltrimethylammonium chloride in water are added, and the mixture is stirred for another 2-6 h at 45° C. At the end of the reaction time, 5-9 ml of water are added, the mixture is cooled down to room temperature and neutralized with 1 M HCl. Then, the product is isolated by precipitation in 80 ml of ethanol. The solids are washed with ethanol, re-dissolved in 20 ml of water and further purified by dialysis. Freeze-drying affords the product as a white solid.

### Example 4

### Synthesis of long-chain 3-(N-alkyl-N,N-dimethylammonio)-2-hydroxyprop-1-yl derivatized phytoglycogen

8.23 mmol of (3-chloro-2-hydroxy-prop-1-yl)dimethylalkyl ammonium chloride (alkyl=lauryl, cocoalkyl, stearyl) are mixed with 0.82 ml of 50% NaOH at 45° C. After stirring for 5 min, 3.33 ml of 20% solution of phytoglycogen in water is added and the mixture is stirred for 6 h at 45° C. The reaction mixture is cooled down to 25° C. and neutralized with 1 M HCl. Then, the product is isolated by precipitation in 50 ml of hexanes. The solids are washed with hexanes, re-dissolved in 20 ml of water and 10 ml saturated NaCl, and further purified by dialysis. Freeze-drying affords the product as a white solid.

### Example 5

### Alkylation of Cationized Phytoglycogen

0.5 g of cationized phytoglycogen with DS=0.88 from Example 4 is dissolved in 10 ml of dry dimethylsulfoxide at 80° C. 0.5 ml of water and 50% NaOH (different amounts, a. 0.021, b. 0.083, c. 0.206, d. 0.495, e. 1.235) are added and stirred vigorously for 10 min. Then, different amounts of alkyl halides (ethyl iodide, benzyl bromide, dodecyl iodide, octadecyl iodide; amounts: a. 0.255 mmol, b. 1.02 mmol, c. 2.55 mmol, d. 6.12 mmol, e. 15.3 mmol) are added. The mixture is stirred for 2 h at 60° C., then it is cooled to room temperature and neutralized with glacial acetic acid. Then, the reaction mixture is extracted several times with diethyl ether and/or hexanes, and re-suspended in saturated aqueous NaCl. After dialysis and freeze-drying, the product is obtained as a white powder. In the case of the dodecyl and octadecyl modifications, the dry product was washed with diethyl ether to remove residual long-chain alcohols.

### Example 6

### Acylation of Cationized Phytoglycogen

0.5 g of cationized phytoglycogen with DS=0.88 from Example 4 are weighed into a glass vial with septum. Through a syringe, 30.5 mmol of neat acid chloride (butyryl chloride, lauroyl chloride) are added drop-wise while stirring. The forming suspension is stirred for another 1 h, and then precipitated by addition of 30 ml of hexanes. The precipitate is washed three times with hexanes, dissolved in 10 ml saturated NaHCO₃ and stirred for 1 h. Excess NaHCO₃ is neutralized with 1M HCl, then 20 ml saturated NaCl are added and the mixture is dialyzed. Oven-drying at 60° C. affords the product as a white powder.

### Example 7

### Silylation of Cationized Phytoglycogen

0.5 g of cationized phytoglycogen with DS=0.88 from Example 4 are oven-dried at 105° C. for 16 h, and dissolved in 10 ml dimethylsulfoxide by heating at 80° C. for 1 h. The reaction vessel is capped with a rubber septum and cooled to 0° C. Triethylamine is added (different amounts: a. 0.166 ml, b. 0.662 ml, c. 1.65 ml, d. 3.97 ml, e. 9.53 ml) followed by dropwise addition of silyl chloride (trimethylsilyl chloride, triethylsilyl chloride; amounts: a. 0.36 mmol, b. 1.46 mmol, c. 3.64 mmol, d. 8.74 mmol, e. 20.74 mmol). The reaction mixture is stirred overnight, then precipitated into acetonitrile. The solids are washed several times with hot acetonitrile, and then dried overnight at 60° C. and 100 mbar.

TABLE 1
Degree of substitution, hydrodynamic
radius and zeta potential for Examples 4-7

| | reagent | | Hydrodynamic | ζ-potential |
|---|---|---|---|---|
| | amount (mol- | | diameter | (DLS) in |
| substituent | eq. of AGU) | DS_{NMR} | (DLS) in D₂O | mV |
| Ethyl | 0.15 | 0.006 | 61.95 | 53.3 |
| Ethyl | 0.6 | 0.083 | 64.81 | 51.4 |
| Ethyl | 1.5 | 0.66 | 64.38 | 51.3 |
| Ethyl | 3.6 | 2.91* | 101.2 | 45.3 |
| Ethyl | 9 | 3.3* | 94.37 | 55.4 |
| Dodecyl | 0.15 | 0.004 | 76.91 | 57.1 |
| Dodecyl | 0.6 | 0.17 | 166 | 33.3 |
| Dodecyl | 1.5 | 0.58* | | |
| Benzyl | 0.15 | 0.052 | 64.57 | 57.9 |
| Benzyl | 0.6 | 0.528 | 70.19 | 57.5 |
| Benzyl | 1.5 | 1.2* | 78.64 | 62.5 |
| Benzyl | 3.6 | 1.58 | 65.61 | 51.6 |
| Benzyl | 9 | 3.6* | 60.45 | 56.5 |
| Butyryl | 6 | 0.02 | 101.7 | 54 |
| Lauroyl | 6 | 0.01 | 120.8 | 52 |
| Triethylsilyl | 0.28 | 0.19* | | |
| Triethylsilyl | 0.7 | 0.45* | | |
| QUAB 342 | 2 | 0.88 | 62.74 | 67.7 |
| QUAB 360 | 2 | 1.02* | 76.58 | 52.6 |
| QUAB 426 | 2 | 0.136 | 165.7 | 57.7 |

*broad NMR peaks, imprecise DS determination.

### Example 8

### Amination of Phytoglycogen with 2-bromoethylamine

200 mg of phytoglycogen were dissolved in 2 mL dimethylsulfoxide, and 250 mg powdered

NaOH slowly added and stirred for 15 min. A solution of 324.9 mg 2-bromoethylamine.HBr in 1.5 mL dimethylsulfoxide was added. After 10 minutes, an additional 0.5 mL dimethylsulfoxide were added and the reaction was stirred for another 4 h at 25° C. After 4 h, 10 mL of water were added to the reaction mixture. The product was precipitated into 28 ml of ethanol, cooled to 0° C., and washed three times with cold ethanol. Drying at room temperature afforded the product as a white solid.

### Example 9

### Conjugation of Aminated Phytoglycogen with Cy5.5-N-hydroxysuccinimide Ester

100 mg of aminated phytoglycogen (prepared according to Example 8) were suspended in 19.8 mL 0.1 M Sodium bicarbonate buffer, pH 8.4. A solution of 1 mg Cy5.5-NHS ester in 2.2 mL dimethylsulfoxide was added and vortexed. The reaction was left to stir at room temperature overnight in the dark. The sample was precipitated by addition of 45 ml cold ethanol. The solids were washed with cold ethanol until the supernatant is colorless, and air-dried to obtain the product as a blue solid.

### Example 10

### Conjugation of Polysaccharide Nanoparticles with Cy5.5-N-hydroxysuccinimide Ester

100 mg of polysaccharide nanoparticles, produced according to Example 1, were suspended in 20 mL of 0.1 M Sodium bicarbonate buffer, pH 8.4. With a temperature probe in a control vial (containing 0.1 M Sodium bicarbonate buffer), the reaction vessel containing the solution was wrapped in aluminum foil and placed on a hot plate at 35° C. 1 mg Cy5.5-NHS ester (Lumiprobe Corp.) was suspended in 4 mL DMF. During a 1 hr period, Cy5.5-NHS ester was added in 1-mL aliquots. The pH of solution was constantly checked before and after addition, adjusting to 8.4 with the addition of a 2 M HCl solution. After the final aliquot of Cy5.5NHS ester in DMF was added, the pH was monitored and adjusted as needed. The reaction was allowed to proceed for 2 hrs further, after which the pH was adjusted to 4.0 with a 2 M HCl solution as aforementioned.

To the acidified solution containing the resulting polysaccharide nanoparticle-Cy5.5 conjugate was added 2 volumes of ethanol. This solution was cooled to 4° C. and centrifuged at 6000 rpm for 15 minutes. After centrifugation, the supernatant was poured off and the pellet was resuspended in 15 mL deionized water. 2 volumes of ethanol were added to the resuspended pellet and it was cooled and centrifuged as before. This was repeated one time further until the supernatant that was poured off was clear and colourless. The pellet was resuspended a final time in 10 mL anhydrous diethyl ether via use of a homogenizer. The resulting conjugate was rendered by evaporating to dryness with trace heat.

### Example 11

### TEMPO Oxidation of Polysaccharide Nanoparticles

50 mg of polysaccharide nanoparticles, produced according to Example 1, was suspended in 15 mL 0.05 M glycine buffer, pH 10.0. The solution was placed in an ice bath to cool to 4° C. for 30 minutes. 0.3 mg (2,2,6,6-Tetramethylpiperidin-1-yl)oxidanyl (TEMPO) and 3.5 mg Sodium bromide were suspended in 250 μL 0.05 M glycine buffer (pH 10). After 30 minutes, both were added dropwise to the polysaccharide nanoparticle solution. 0.08 mL Sodium hypochlorite was subsequently added to the polysaccharide nanoparticle solution and it was subsequently sealed for reaction. Oxidation was permitted to continue for 26 hrs. Oxidation was terminated by the addition of 40 μL of ethanol. The oxidation was stirred for a further 30 min at room temperature.

The solution containing oxidized polysaccharide nanoparticles was removed to dialysis bagging (Spectrum Laboratories, Inc.; MWCO 12-14,000) to exchange against deionized water for 2 days. Afterwards, the residual solution from the dialysis bagging was lyophilized to dryness to render the conjugate as a dry compound.

### Example 12

### Conjugation of TEMPO-Oxidized Polysaccharide Nanoparticles to Amphotericin B

Prior to reaction, a solution containing 1-ethyl-3-(3-dimethylaminopropyl)carbodiimide (EDC) and 2-(N-morpholino)ethanesulfonic acid (MES), pH 5.5, was created for use (13.5 mL 0.5 M MES buffer, pH 5.5; 270 μL EDC). 27 mg of oxidized polysaccharide nanoparticles, produced by TEMPO oxidation as previously outlined, was dissolved in 10 mL of the aforementioned EDC/MES solution. The dissolved polysaccharide nanoparticles were permitted to react with 9 mg amphotericin B (in 3.5 mL EDC/MES) at room temperature for 2 hrs. Thereafter, the reaction mixture was brought to 37° C. to react for a further 24 hrs.

After 24 hrs, the conjugation reaction solution was transferred to dialysis bagging and dialysed against deionized water for 2 days. The resulting solution contained in the dialysis bagging was lyophilized to dryness to render the conjugate.

The product was analyzed using UV-Vis spectroscopy and it was found that 1 mg of conjugate contained 73 μg of amphotericin B. This corresponds to a DS of 0.015.

### Example 13

### Cytotoxicity of Glycogen/Phytoglycogen in Cell Cultures

The effects of the glycogen/phytoglycogen nanoparticles on cell viability was analyzed to assess cytotoxicity of the particles. Glycogen/Phytoglycogen nanoparticles were extracted from rabbit liver, mussels, and sweet corn using cold-water and extracted as described in Example 1.

Cell lines used: rainbow trout gill epithelium (RTG-2).

To measure changes in cell viability two fluorescence indicator dyes were used, alamar blue (ThermoFisher) and CFDA-AM (Thermofisher); these dyes measure cell metabolism and membrane integrity respectively. For these dyes, more fluorescence indicates more viable cells.

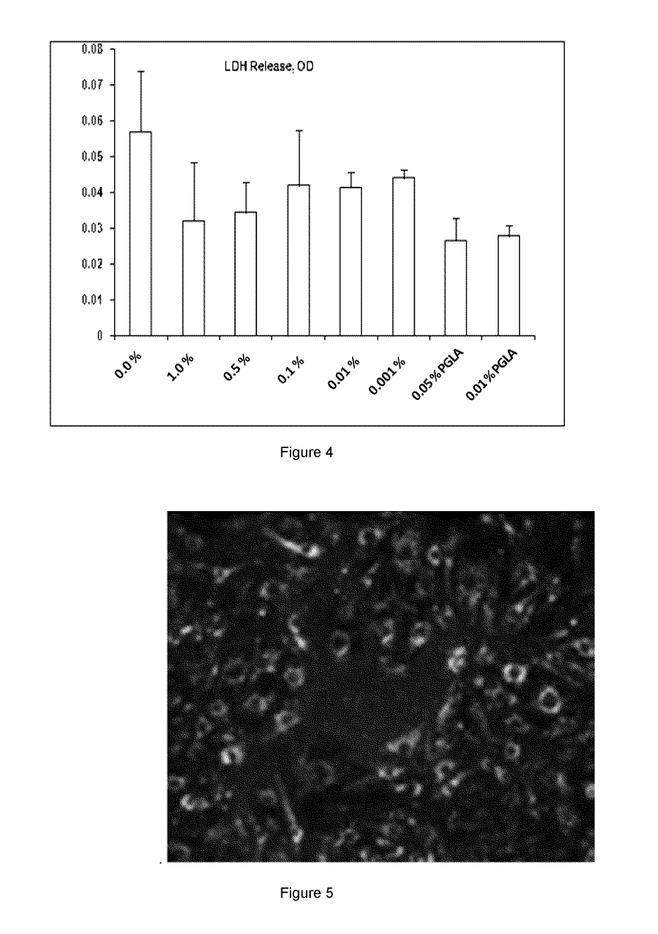

The results are presented in FIGS. 3 and 4. None of the assays detected any of cytotoxicity effects in cells after 48 hrs incubation in the presence of phytoglycogen at concentrations of 0.1-10 mg/mL.

### Example 14

### Cellular Uptake of Glycogen/Phytoglycogen Nanoparticle Compositions

Fluorescence microscopy was performed of normal murine endothelial cells exposed to phytoglycogen nanoparticles conjugated to Rhodamine B (orange fluorescence). As shown in FIG. 5, the nanoparticles accumulated only in the cytoplasm.

Fluorescence microscopy was performed on white blood cells exposed to phytoglycogen nanoparticles conjugated to Rhodamine B.

Two milliliters of blood from the wing vein was collected by a 5-mL syringe containing 50 μg/mL of heparin to prevent clotting. Peripheral blood mononuclear cells were isolated by density-gradient. Briefly, 2 mL of heparinized blood mixed with an equal volume of PBS was added carefully onto the surface of 2 mL of Histopaque 1083 (Sigma-Aldrich) in a 10-mL conical tube and then was subject to centrifugation at 500×g for 20 min at room temperature. After centrifugation, mononuclear-containing cells in the interface between the first layer and Histopaque 1083 medium were collected. After being washed with 5 mL of 38° C. heated PBS 3 times by centrifugation at 500×g for 5 min, the pellet was resuspended with the complete medium containing 90% RPMI 1640 (Invitrogen) and 10% fetal bovine serum.

P. aeruginosa PAO1 was cultured in Tryptic Soy Broth (TSB) medium at 32° C. on a shaker at 180 rpm. Bacterial cells were harvested from overnight culture by centrifugation at 3000×g for 15 min. and then resuspended in TSB to a density of ca. 10⁹ cells/ml.

Monocyte cell suspensions were mixed with nanoPG-RhodamineB (final conc. ca. 0.1%) and/or bacteria (to a final bacterial cell concentrations ca. 10⁸ cells/ml) and the mixtures were incubated at 38° C. for 2 hrs prior to CLSM investigation.

As shown in FIG. 6 (the arrows P indicate phagosomes with bacterial cells and PGRhodamineB) the phytoglycogen nanoparticles conjugated to Rhodamine B were taken up by the monocytes and localized in phagosomes. In this experiment, the monocytes were activated by the bacteria and then internalized both the bacteria and nanoPG-RhodamineB. As a result, RhodamineB was co-localized with bacteria in the phagosomes. In contrast, when monocytes were not stimulated by exposure to bacteria, there was no internalization and accumulation of nanoPG-RhodamineB by monocytes. This indicates that nanoPG-RhodamineB did not activate monocyte phagocytosis and, therefore, will not be cleared from the blood stream by monocytes.

### Example 15

### Anti-Infective Properties of Cationized Phytoglycogen

Anti-infectives limit or prevent the spread of infection. Agents inhibiting cell growth or causing cell death have potential as anti-infectives. Minimum inhibitory concentration (MIC) assays were done to evaluate antibacterial properties of cationized phytoglycogen. The MIC is defined as “the lowest concentration of an anti-infective agent that prevents visible growth of a micro-organism in an agar or broth dilution susceptibility test”.

MIC assessments against bacteria were performed following broth micro-dilution in accordance with the Clinical and Laboratory Standards Institute document M07-A9: Methods for Dilution Antimicrobial Susceptibility Tests for Bacteria that Grow Aerobically; Approved Standard. A Gram-positive organism, Bacillus subtilis 168, and two Gram-negative organisms, Escherichia coli AB264 (K-12) and Pseudomonas aeruginosa PAO1 were assessed. Long-term stock cultures of organisms were maintained at −80° C. in glycerol (15% vol/vol). Stocks were revived by sub-culturing onto trypticase soy agar plates, grown for 18 hrs at 37° C. and then stored at 4° C. for up to one week. Overnight cultures were grown by inoculating Mueller-Hinton broth with 3-4 colonies from stock plates and incubating for 20-24 hrs at 37° C. and 150 rpm. Overnight cultures were used to inoculate fresh Mueller-Hinton broth (2% vol/vol). Cultures were then grown to the mid-exponential phase, approx. 2-4×10⁷ CFU.ml⁻¹, at which point cultures were used to prepare inocula for MIC plates. Sterile solutions of gamma-irradiated native phytoglycogen and cationized phytoglycogen were prepared by reconstitution and dilution as required in sterile Mueller-Hinton broth. A side-by-side comparison was performed using matched filter-sterilized materials MIC was assessed at final in-assay concentrations of 100-1000 μg native or cationized phytoglycogen.ml⁻¹, increasing in increments of 100 μg.ml⁻¹. Negative growth controls comprised sterile medium. Positive growth controls contained inoculated medium. The inoculum was prepared immediately prior to use by diluting in sterile Mueller-Hinton broth. Further dilution in the assay yielded a final in-assay cell density of 5×10⁵ CFU.ml⁻¹. Plates were incubated at 37° C. for 20 h, at which points wells were scored for growth. MIC was recorded as the lowest concentration of an agent which resulted in no growth (optically clear). MIC assays were performed in triplicate within each experiment and were repeated twice to confirm data (n=6). Cationized phytoglycogen, prepared according to the alternative procedure detailed in Example 3, was also assessed in double-dilution increments from 19 to 10 000 μg.ml⁻¹. MIC assays were performed in single replicate and were repeated thrice to confirm data (n=3).

MIC assay was done to evaluate the antifungal activity of cationized phytoglycogen against the yeast Candida utilis ATCC9950. Broth micro-dilution assay was performed in accordance with the Clinical and Laboratory Standards Institute document M27-A2 Reference Method for Broth Dilution Antifungal Susceptibility Testing of Yeasts; Approved Standard—Second Edition. Long-term stock cultures of C. utilis were maintained at −80° C. in glycerol (15% vol/vol). RPMI 1640 medium (Sigma-Aldrich; Canada), containing no sodium bicarbonate, supplemented with 0.165 M MOPS and adjusted to a pH of 7.0, was used for the assay. Sterile solutions of gamma-irradiated native phytoglycogen and cationized phytoglycogen (Example 2) were prepared by reconstitution and dilution as required in sterile broth. A side-by-side comparison was performed using matched filter-sterilized materials. MIC was assessed at concentrations of final in-assay concentrations from 30 to 100 μg cationized phytoglycogen.ml⁻¹ RPMI 1640, increasing in 10 μg.ml⁻¹ increments. Negative growth controls comprised sterile medium. Positive growth controls contained inoculated medium. The inoculum was prepared immediately prior to use. An overnight culture of C. utilis ATCC9950, grown in tryptic soy broth (35° C., 150 RPM), was diluted to yield a final in-well concentration of 0.5×10³ CFU.ml⁻¹ in all wells except the negative growth control wells. Upon inoculation, plates were incubated statically for 48 h at 35° C. At the termination of the assay wells were scored for growth. The MIC was recorded as the lowest concentration of an agent which resulted in no growth (optically clear). Experiments were performed as in-assay triplicates and repeated three times (n=9).

Both B. subtilis 168 and E. coli K-12 were susceptible to cationized phytoglycogen, with Inhibition of cell growth at concentrations of 200 and 300 μg.ml⁻¹ respectively. At all tested concentrations, native phytoglycogen did not cause growth inhibition. Growth of P. aeruginosa PAO1 was not affected by the cationized phytoglycogen at the concentration tested. This may be due to the robust cell wall architecture and adaptability of this organism, and does not preclude the potential inhibitory effects at higher concentrations.

Similarly, concentrations of ≥60 μg cationized phytoglycogen.ml⁻¹ resulted in growth inhibition of the yeast C. utilis ATCC9950. At all tested concentrations, native phytoglycogen did not cause growth inhibition.

The alternate synthesis protocol (described in Example 3) was also used to generate cationized phytoglycogen, substituted to varying degrees of substitution (DS) and which was found to be important for growth inhibition. The MIC of this cationized phytoglycogen, matched to that obtained using the Example 3 synthesis protocol and having a similar DS of 0.7, remained relatively similar against B. subtilis 168 with a value of 312.5 μg.ml⁻¹. The MIC against the two Gram-negative bacteria, E. coli K-12 and P. aeruginosa PAO1 were substantively altered, both having values of 10 000 μg.ml⁻¹. Increasing the DS to a value of 1.34 lowered these MIC values to 2500 and 312.5 μg.ml⁻¹ respectively. The increased sensitivity shown by P. aeruginosa PAO1 is likely due to increased interaction between the core region of lipopolysaccharide in the outer surface of the bacterial cells, and which carries a greater net negative charge in P. aeruginosa PAO1 than in E. coli, with cationized phytoglycogen bearing a greater net positive charge.

### Example 16

### Double Modification of Phytoglycogen Improves the Growth Inhibition of Properties of Cationized Phytoglycogen

Phytoglycogen was modified according to Examples 5-7 to bear both cationic groups and other substituents. MIC values were established according to the protocol previously described in Example 15. Experiments were performed as single in-assay replicates and repeated three times (n=3). MIC values with a four-fold or greater change relative to the MIC of the single-modification cationized) phytoglycogen represented a statistically-significant change in MIC value. Only those double-modifications to cationized phytoglycogen combinations which caused a four-fold change relative to the MIC of the single-modification cationized phytoglycogen were indicative of resulting in statistically significant improvement (MIC value was statistically significantly less).

The two modifications from the assessed panel that resulted in statistically significant enhancement of the MIC of cationized phytoglycogen against B. subtilis 168 were the butyryl or QUAB426 substituents. In both instances, the MIC was reduced by 8-fold concentration when the reagent was used at molar equivalent ratios of 6 and 2 respectively during the synthesis protocol.

No significant enhancement was found for the Gram-negative E. coli K-12, whereas a number of the modifications resulted in reductions in MIC when tested against P. aeruginosa PAO1. Notably, the benzyl modification resulted in 8-fold reductions when employed at molar equivalent ratios of 0.15 and 9, and 32-fold reduction when employed at molar equivalent ratios of 0.6, 1.5 and 3.6. The ethyl modification resulted in 8-fold reductions when employed during synthesis at molar equivalent ratios of 0.15, 0.6 and 9. The butyryl and lauryl modification, at molar equivalent ratios of 6, both resulted in 8-fold reductions in MIC as well. Generation of a trimethylsilyl double modification reduced MIC 8-fold (reagent used at 0.07, 0.28 molar equivalent ratio), and four-fold (4.3). Double-modification of cationized phytoglycogen to bear triethylsilyl groups reduced MIC by 8-fold (molar equivalent ratio of 0.07).

### Example 17

### Cationized Phytoglycogen Enhances the Susceptibility of Planktonic Cells to Antibiotics

MIC assays as defined in Example 15 were performed in sterile and untreated 96 well microtitre plates according to the broth micro-dilution technique described in either the Clinical and Laboratory Standards Institute document M07-A9: Methods for Dilution Antimicrobial Susceptibility Tests for Bacteria that Grow Aerobically; Approved Standard and document M27-A2 Reference Method for Broth Dilution Antifungal Susceptibility Testing of Yeasts; Approved Standard—Second Edition, with the exception that, where indicated, medium was additionally supplemented with native or cationized phytoglycogen. Assessments done using C. utilis ATCC9950, B. subtilis 168 and E. coli AB264 employed native or cationized phytoglycogen at concentrations ⅛, ¼, ½ their respective MIC values for cationized phytoglycogen. Assessments of the opportunistic pathogen P. aeruginosa PAO1 were made at final in-assay concentrations of 0.5, 1 or 10 mg native or cationized phytoglycogen.ml⁻¹. Experiments were performed as in-assay duplicates, and were repeated a minimum of three times (n=6).

Antibiotics screened were representative of a number of different classes and are summarised in Table 2. MIC assays were performed in duplicate within each experiment and were repeated a minimum three times to confirm the data (n=6). MIC values with a four-fold or greater change relative to the non-supplemented medium MIC represent a statistically-significant change in MIC value. Only those antibiotic-cationized phytoglycogen combinations which caused a four-fold change relative to the non-supplemented medium MIC were deemed as indicative of resulting in potentiation (MIC value was less), or tolerance (MIC value increased).

For all tested organisms, supplementation with the native phytoglycogen resulted in MIC values which remained the same as or showed a two-fold change relative to the MIC values obtained in non-supplemented medium. That is, native phytoglycogen did not affect the MIC of representative examples from diverse classes of antibiotics. This highlights the potential of native phytoglycogen as a neutral platform for the development of tailored nanotechnology targeted against micro-organisms e.g. to deliver high and localised doses of conjugated antibiotics.

TABLE 2
Summary of antibiotics employed in sensitization to antibiotics assay

| antibiotic | | | range of in-assay concentration (μg · ml⁻¹) | | | |
|---|---|---|---|---|---|---|
| class | antibiotic | abbvn. | B. subtilis | E. coli | P. aeruginosa | C. utilis |
| [symbol] minoglycoside | gentamicin | GEN | 3.9 × 10⁻³-2 | 1.95 × 10⁻³-1 | 0.0625-32 | — |
| | tobramycin | TOB | — | — | 0.0625-32 | — |
| β-lactam | ceftazidime | CAZ | — | — | 2 × 10⁻³-32 | — |
| | penicillin G | PNG | 4.8828 × 10⁻⁴-0.25 | 0.5-256 | 48-25000 | — |
| | carbenicillin | CAR | 7.8125 × 10⁻³-4 | 0.0625-32 | 2-1024 | — |
| | ampicillin | AMP | 2.4414 × 10⁻⁴-0.125 | 0.015625-8 | 2-1024 | — |
| glycopeptide | vancomycin | VAN | 3.9 × 10⁻³-2 | 1-512 | 2-1024 | — |
| [symbol] uoroquinolone | ciprofloxacin | CIP | 1.95 × 10⁻³ -1 | 2.44 × 10⁻⁴-1.95 × 10⁻³ | 0.00195-1 | — |
| quinolone | nalidixic acid | NAL | — | — | 2-1024 | — |
| macrolide | erythromycin | ERY | 1.2207 × 10⁻⁴-0.0625 | 0.03125-16 | 2-1024 | — |
| amphenicol | chloramphenicol | CHL | 0.03125-16 | 0.25-128 | 2-1024 | — |
| tetracycline | tetracycline | TET | — | — | 2-1024 | — |
| [symbol] minocoumarin | novobiocin | NOV | — | — | 4-2048 | — |
| lipopeptide | polymyxin B | PMB | — | — | 0.0625-32 | — |
| polyene | amphotericin B | AMB | — | — | — | 0.03125-16 |

[symbol]

indicates data missing or illegible when filed

In contrast, supplementation with cationized phytoglycogen resulted in several-fold changes to MIC values of select antibiotics for all assayed organisms (Tables 3, 4). In all instances, these value changes were fold reductions i.e. cells were more susceptible to antibiotics. Sensitization to an antibiotic was also concentration-dependent, and at low concentrations this was lost (Table 3) or reduced (Table 4). As expected, when cationized phytoglycogen was employed at sub-MIC concentrations (Table 3), the potentiation effects were less than when employed at higher concentrations (Table 4). In addition, not all antibiotic-cationized phytoglycogen combinations showed sensitization; this was both concentration-dependent and also related to the target of the antibiotic and the organism used in the screen. That is, there exists optimal combinations of antibiotic and cationized phytoglycogen which need to be established empirically. Overall, assessment across all organisms and cationized phytoglycogen-antibiotic combinations tested, indicated 13 out of 14 different classes of antibiotics showed statistically significant fold-reductions.

TABLE 3
Sub-MIC cationized phytoglycogen sensitizes micro-organisms to antibiotics.

| | Candida utilis | | | Bacillus subtilis | | | Escherichia coli | | |
|---|---|---|---|---|---|---|---|---|---|
| Antibiotic | *CP_{0.5} | CP_{0.25} | CP_{0.125} | CP_{0.5} | CP_{0.25} | CP_{0.125} | CP_{0.5} | CP_{0.25} | CP_{0.125} |
| amphotericin B | ↓4 | 1 | 1-2 | — | — | — | — | — | — |
| gentamicin | — | — | — | ↓4-8 | ↓2-4 | 1-2 | ↓4-8 | 2 | 1-2 |
| ciprofloxacin | — | — | — | ↓2-4 | 2 | 1 | 1-2 | 1-2 | 1-2 |
| penicillin G | — | — | — | ↓16-32 | ↓8-16 | 1-2 | ↓8 | ↓4-8 | 1-2 |
| carbenicillin | — | — | — | ↓4-8 | 2 | 1 | ↓8-16 | ↓4-8 | 1-2 |
| ampicillin | — | — | — | 2 | 1 | 1 | 1-2 | 1-2 | 1-2 |
| vancomycin | — | — | — | 1-2 | 1 | 1 | ↓4-16 | ↓2-4 | 1-2 |
| erythromycin | — | — | — | ↓2-4 | ↓2-4 | 1-2 | 1-2 | 1-2 | 1-2 |
| chloramphenicol | — | — | — | | ↓2-4 | 1 | 1-2 | 1-2 | 1-2 |

*CP denotes supplementation with cationized phytoglycogen and the number indicates the sub-MIC strength at which supplementation was done e.g. CP_{0.5} indicates half-strength of MIC. Numbers in the table indicate fold-change reductions in MIC relative to the MIC (non-supplemented medium).

TABLE 4
Supplementation with cationized phytoglycogen enhances
sensitivity of P. aeruginosa to multiple and diverse antibiotics

| | | ↓ Fold-change in MIC | | |
|---|---|---|---|---|
| | MIC | (cationized phytoglycogen) | | |
| Antibiotic | (μg · ml⁻¹) | MIC_{.5 mg/ml} | MIC_{1 mg/ml} | MIC_{10 mg/ml} |
| gentamicin | 0.5 | 2 | 2 | 1 |
| tobramycin | 0.25 | 2 | 2 | 1 |
| ceftazidime | 2 | ↓ 8 | ↓ 32-64 | ↓ 4-8 |
| ciprofloxacin | 0.125 | ↓ 2-4 | ↓ 8 | 2 |
| penicillin G | 12500 | ↓ 16-32 | ↓64-128 | ↓ 2-4 |
| carbenicillin | 64 | ↓ 16-32 | ↓ 32 | ↓ 8-16 |
| ampicillin | 1024 | ↓ 8 | ↓ 8-16 | 2 |
| vancomycin | 1024 | ↓ 2-4 | ↓ 16 | ↓ 4 |
| nalidixic acid | 1024 | ↓ 4-32 | ↓ 16-32 | ↓ 8-16 |
| erythromycin | 128 | ↓ 4 | ↓ 8 | 1-2 |
| chloramphenicol | 64 | ↓ 4 | ↓ 8 | 2 |
| tetracycline | 16-32 | ↓ 8 | ↓ 8 | 2 |
| novobiocin | 2048 | ↓ 4-16 | ↓ 16-32 | ↓ 4 |
| polymixin B | 0.125-0.25 | 1-2 | ↓ 2-4 | 2 |

* MIC_{0.5}, MIC₁, and MIC₁₀ refer to the fold-change reduction in MIC values obtained when supplemented with 0.5, 1 or 10 mg cationized phytoglycogen · ml⁻¹ Mueller-Hinton Broth. Values are reported as fold change with respect to the MIC values obtained in non-supplemented Mueller-Hinton Broth.

Cationized phytoglycogen sensitized bacteria and yeast to the action of diverse classes of antibiotics and lower concentrations of antibiotics were required to achieve growth inhibition in the assay. Such changes to MIC values are also indicative of potential underlying mechanisms of action and include perturbation of the cell permeability barrier, and also responsive changes resulting in a more hydrophobic cell surface. In addition, changes to cell properties such as permeability may also render the cells more sensitive to components of the immune system. Treatment of cells with permeabilizers such as EDTA has demonstrated enhanced action of lysozyme, a component of the innate immune system which attacks the bacterial cell wall, resulting in cell lysis. Accordingly, cationized phytoglycogen nanoparticles may be used as co-therapeutic whereby infectious agents such as bacteria and yeast are rendered more tractable to chemotherapeutic regimens such as antibiotics and also the host immune system.

### Example 18

### Synthesis of Antibiotic-Phytoglycogen Conjugates with Antimicrobial Activity

The method for conjugation of amphotericin B to phytoglycogen nanoparticles is detailed in Example 12.

The activity of the amphotericin B-phytoglycogen conjugate (PHG-AMB) was assayed against a yeast, Candida utilis ATCC9950, using a modified broth microdilution assay (CLSI M27-A2; Example 15). RPMI 1640 medium (Sigma-Aldrich; Canada), containing no sodium bicarbonate, supplemented with 0.165 M MOPS and adjusted to a pH of 7.0, was used for the assay. Stocks of 1600 μg AMB/ml DMSO were stored at −80° C. and diluted prior to use. 500 μg PHG-AMB.ml⁻¹ RPMI 1640 was prepared fresh on the day of assay. Briefly, doubling dilutions of 16 μg AMB.ml⁻¹ RPMI 1640 and 500 μg PHG-AMB.ml⁻¹ RPMI 1640 were prepared by serial two-fold dilution in RPMI 1640. Controls comprised AMB supplemented with phytoglycogen adjusted to mimic the changing concentration of PHG-AMB in the wells. Negative growth control wells contained sterile medium. Positive growth control wells contained medium or medium supplemented with phytoglycogen. The MIC was recorded as the lowest concentration of an agent which resulted in no growth (optically clear) in a well. Assays were performed as in duplicate replicate with a minimum of three independent replicates.

MIC values of 0.5 μg AMB.ml⁻¹ in RPMI 1640 and RPMI 1640 supplemented with phytoglycogen were obtained following growth of C. utilis ATCC9950 for 48 h at 35° C. That is, consistent with the previous observations on antibacterial antibiotics, phytoglycogen had no impact on the MIC of an antifungal.

The conjugate PHX-AMB displayed growth inhibitory properties with a MIC of 31.25 μg PHG-AMB.ml⁻¹. UV-Vis spectrophotometry was used to determine that the conjugate contained 73 μg of amphotericin B per mg conjugate, this yielded an absolute MIC value of 2.28 μg amphotericin B.ml⁻¹. That is, amphotericin B could be conjugated to phytoglycogen and remain active, with some reduction in MIC, and highlighted that phytoglycogen may function as a nanoparticle delivery system for antibiotics.

### Example 19

### Cationized Phytoglycogen Negatively Affects Production of Pyocyanin, a Virulence Factor which is Tightly Regulated Via Quorum Sensing Pathways

Pyocyanin is a pigment produced by many strains of P. aeruginosa and serves diverse roles as a virulence factor, in redox processes and as a terminal signal in quorum sensing pathways. Importantly, the production of pyocyanin is tightly regulated via a hierarchy of quorum sensing signaling systems. These systems, and others, form an intricate regulatory network which together govern other key phenotypes critical for the virulence of P. aeruginosa and its ability to cause acute and chronic infection. The readily discernible signature blue-green colour of pyocyanin in culture has made this a frequent choice in initial screens to assess for interference with quorum sensing systems and consequent alterations in virulence factor production. While antibacterial activity is desirable, the ability to attenuate virulence is also an asset. Macrolide antibiotics such as azithromycin have been utilised as a co-therapeutic in the treatment of P. aeruginosa infections within the respiratory tracts of patients with cystic fibrosis, not as an antibiotic but as a means to regulate virulence and pathogenicity.

Pyocyanin production was assayed and quantified according to previously known methods with minor modifications. P. aeruginosa cultures were grown in Mueller-Hinton broth at 37° C. and 150 rpm in the presence of varying concentrations of native phytoglycogen or cationized phytoglycogen. At 20 h, 2 ml of culture were extracted by vigorous mixing with 1.2 ml of chloroform. After phase separation, the chloroform phase was transferred to a fresh tube, 0.4 ml of 0.2 N HCl was added and the phases were mixed vigorously. Once the phases separated, 200 μl aliquots of the HCl phase were transferred to the wells of a 96 well plate and the A₅₅₀ measured with a BioTek EL800 plate reader; values were converted to ug pyocyanin.ml⁻¹ using a calibration plot. Experiments were performed in triplicate and repeated in independent triplicate experiment (n=9).

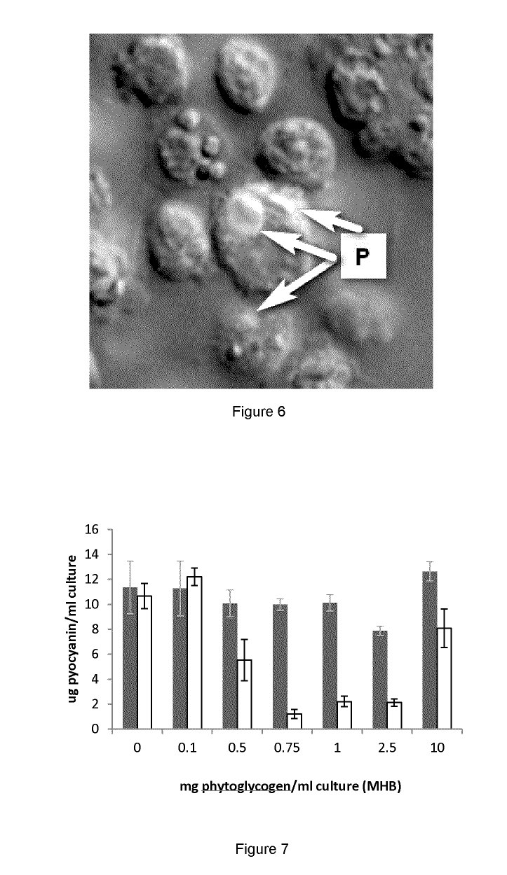

Quantitation of pyocyanin production following 20 h incubation in the presence of native or cationized phytoglycogen revealed that only cationized phytoglycogen affected pyocyanin production (FIG. 7). At concentrations of 0.5 to 2.5 mg.ml⁻¹ of cationized phytoglycogen, reductions of 50-90% in pyocyanin production were measured relative to non-supplemented medium. At 10 mg.ml⁻¹ there was a ca. 75% restoration of pyocyanin production. Curiously, when the upper limit was exceeded pyocyanin production did not exceed that in non-supplemented conditions.

That is, Incubation of P. aeruginosa with cationized phytoglycogen affected production of the virulence factor, pyocyanin. Moreover, this resulted in a U-shaped concentration dependent response, indicating lower and upper thresholds of efficacy, and a concentration range of optimal efficacy with respect to the ability of the modified nanoparticles to impair production of the virulence factor pyocyanin.

### Example 20

### Cationized Phytoglycogen Negatively Impacts Bacterial Motility, a Process which is Important for Bacterial Migration, Colonization and Infection

Pseudomonas aeruginosa displays three forms of motility—swimming, swarming and twitching—all of which contribute to the organism's ability to cause infectious disease and are important for migration, attachment and colonization, as well as biofilm formation and maturation, and dispersal from a biofilm population or nidus of infection.

A modified swimming motility assay was used to assess for inhibition of swimming motility of P. aeruginosa by cationized phytoglycogen. Stocks were revived by sub-culturing into modified M9 and grown for 20-24 h (150 rpm, 37° C.). Modified M9 medium was prepared as follows—20 mM NH₄.Cl, 12 mM Na₂HPO₄, 22 mM KH₂PO₄, 8.6 mM NaCl, 0.5% wt/vol casamino acids. The medium was sterilized by autoclaving at 121° C. for 30 min and, after cooling to ca. 45° C., supplemented with final concentrations of 11 mM glucose, 1 mM MgSO₄, 1 mM CaCl₂.2H₂O. Motility plates were prepared on the day using mM9 medium solidified with 0.3% (wt/vol) Bacto agar (Becton-Dickson; Fisher Scientific, Canada). After autoclaving, cooled agar was supplemented with the above indicated supplements and also 0.1, 0.5, 0.75, 1.0, 2.5 or 10.0 mg sterile native or cationized phytoglycogen.ml⁻¹ medium. The control comprised swim agar with no added phytoglycogen. 25 ml aliquots were poured into sterile petri dishes, while working in a biological safety cabinet under laminar flow conditions, and allowed to set with lids open (1 h). After 1 h the plates were stab-inoculated with the overnight culture to a few mm below the surface of the agar and then incubated without inversion (37° C., 24 h). At the termination of the growth period, three diameter measurements were recorded per swim zone and used calculate the spread of the surface area of the swim zones. Experiments were performed as in-assay triplicates and repeated three times (n=27).

Swarming motility of P. aeruginosa was measured using swarm plate agar made with modified M9 medium containing 0.5% wt/vol agar. Sterile native or cationized phytoglycogen was added to achieve final concentrations of 0.1, 0.5, 0.75, 1.0, 2.5 or 10.0 mg.ml⁻¹ medium. The control comprised swarm agar without supplementation. Working in a biosafety cabinet, 20 ml aliquots were poured into sterile petri dishes and allowed to set with the lids open. After 15 min, the agar was allowed to set for a total of 60 min. An inoculum was prepared by harvesting cells from a culture of P. aeruginosa PAO1 grown for 24 h in modified M9 medium at 37° C. and 150 rpm. Cells were pelleted and the pellet was resuspended and washed twice in sterile 0.9% wt/vol NaCl. The washed cell pellet was resuspended to a final OD₆₀₀ of 3.0 units. Plates were inoculated by pipetting 3 μl of the inoculum onto the centre of the plate. Plates were left at room temperature for 2 h, prior to incubating at 37° C. for 24 h. After 24 h, observations were made and the plates were incubated for a further 24 h at room temperature. At the termination of the experiment, observations and images were recorded. Three measurements of diameter spread (mm) were made on each plate (n=27). Experiments were performed in triplicate and repeated in independent triplicate experiment (n=27).

Twitching motility was assayed in Luria-Bertani medium (Becton-Dickson; Fisher Scientific, Canada) supplemented with 1% (wt/vol) bacteriological agar (Becton-Dickson: Fisher Scientific, Canada). Where appropriate, the twitching motility agar was cooled to ca. 45° C. and sterile native or cationized phytoglycogen was added to achieve final concentrations of 0.1, 0.5, 0.75, 1.0, 2.5 or 10.0 mg phytoglycogen.ml⁻¹. The control comprised twitch agar without supplementation. Working in a biosafety cabinet, 10 mL aliquots of twitching motility agar were dispensed into sterile petri dishes and allowed to set, with lids closed, for 1 h. P. aeruginosa PAO1 stocks were revived by sub-culturing onto tryptic soy agar plates (Becton-Dickson; Fisher Scientific, Canada) and grown for 20 h at 37° C. An inoculum was made using a sterile pipette tip to scrape several colonies from the surface of the tryptic soy agar plates and stirring these into a thick slurry. The slurry was used to stab inoculate the centre of each motility assay plate all the way through to the base of the plate. Plates were incubated at 37° C. (48 h). Experiments were performed in triplicate replicate, three measurements made on each assessment, and repeated independently three times (n=27). Due to opacity at high concentrations of phytoglycogen, the twitch zone at the agar:plate interface was re-measured after excision of the overlying agar. Values were within ±1 mm.

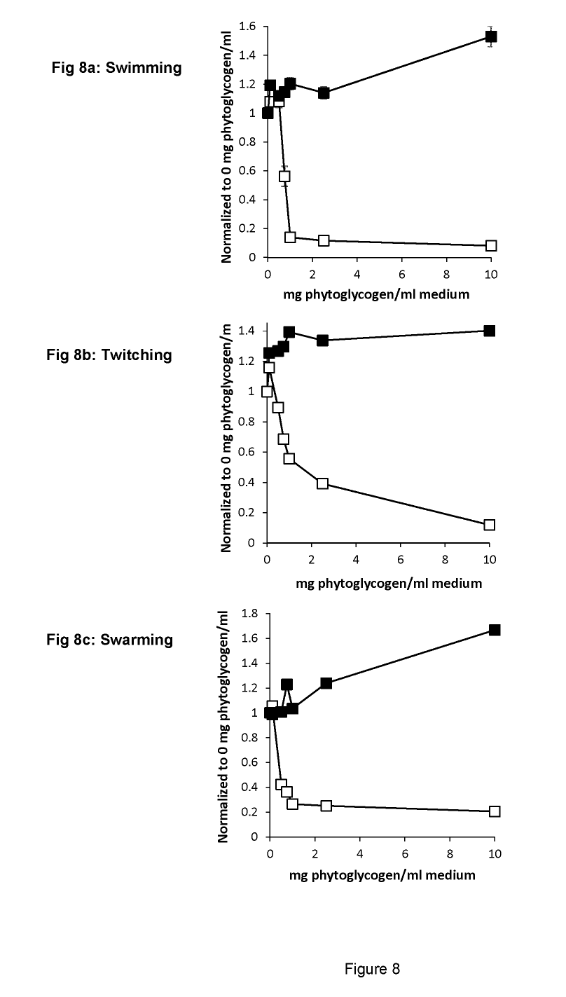

For all three forms of motility, cationized phytoglycogen but not the native non-derivatized phytoglycogen (FIG. 8). Moreover, this was shown to be concentration-dependent, and increasing concentrations of cationized phytoglycogen had greater effect on impeding motility.

Supplementation with phytoglycogen or low concentrations of cationized phytoglycogen resulted in moderate (<10% increase) to high (>20%) increased motility (FIG. 8), signifying potential application limits since swarming and twitching motility have been positively correlated with biofilm formation and growth. However, this was not supported when biofilm formation was assayed (refer to Example 22 for further detail). It is probable that the limitation of efficacy is related to the assay method and that motility will be impeded at lower concentrations than found here. The underlying cause is not understood, yet the incredible water-retaining ability of phytoglycogen is likely of consequence to cells on air-exposed agar plates.

At concentrations of 0.75 mg.ml⁻¹ cationized phytoglycogen clearly impedes all forms of bacterial motility. Since bacterial motility is important for a number of processes which contribute to the spread and progression of infectious disease we also hypothesize that cationized phytoglycogen will be able to act as an anti-infective which affects motility and motility-based processes.

### Example 21

### Native Phytoglycogen Limits the Formation and Accretion of Biofilms

Biofilms are sessile communities of microorganisms in which cells adhere to one another and also, often, to a surface. Members of the community may be drawn from viruses, bacteria, yeast, fungi, algae, protozoa, nematodes. The biofilm is typically encased within a matrix, comprised of extracellular polymeric substances. This is typically produced by the biofilm, but may also incorporate materials from an exogenous source.

Biofilms are present in the natural environment, and are common in hospitals and industrial settings. Biofilms can form on living and non-living surfaces, including native tissues and medical devices. Biofilm communities are more persistent and recalcitrant than free-swimming planktonic cells. Observed differences include decreased susceptibility to anti-infectives and other inimical agents, reduced predation and invasion, and evasion of components of the immune response. That is, once an infectious agent adopts a biofilm mode of growth, clearance or treatment of the infectious agent is more difficult. In cases where microorganisms succeed in forming a biofilm on or within a host, including human hosts, chronic and untreatable infection can result. It is therefore desirable to be able to both limit and control the formation and growth of biofilms.

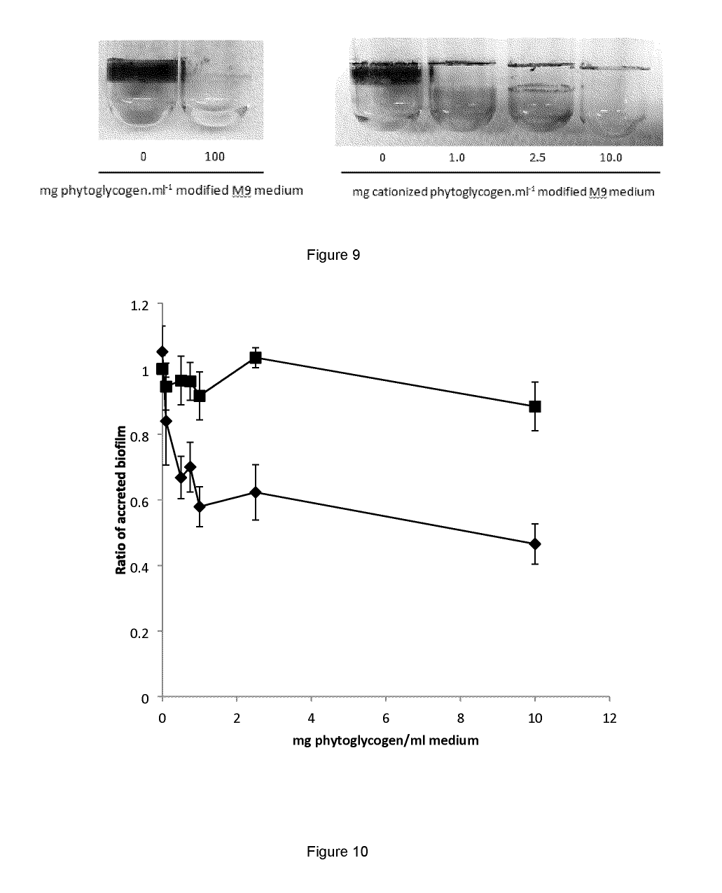

P. aeruginosa PAO1 stocks were revived by sub-culturing P. aeruginosa PAO1 in modified M9 or King's A medium (22-24 h, 37° C., 150 rpm). Modified M9 medium was prepared as described in Example 19 with the exception that no agar was added; King's A medium contained 2% proteose peptone, 1% K₂SO₄, 0.164% MgCl₂, 1% glycerol. Clean, sterile glass tubes (18×150 mm) were used for the assay. Stock media solutions of medium or medium supplemented with sterile native phytoglycogen were prepared. The stock solutions were inoculated with the pre-grown culture (1:2000 dilution), mixed well and 2 ml aliquots transferred to sterile tubes. The tubes were incubated for 20 h (37° C., 150 rpm). Accumulated biofilm was visualized by staining with Hucker's crystal violet (final in-assay concentration of 0.1% wt/vol), for 15 min, at room temperature. Excess stain was removed by decanting and then profuse rinsing with water to remove unretained dye. The tubes were inverted and air dried. The accreted biomass retained stain and was seen as a purple-coloured mass on the walls of the tube (FIG. 9). Images were recorded and archived. Retained stain was solubilised with 33% acetic acid (vol/vol) and quantified spectrophotometrically (A=570 nm) using a Perkin Elmer Lambda 25 UV/Ms spectrophotometer. Experiments were performed in triplicate assay and repeated as independent triplicate experiments (n=9).

The ability of native phytoglycogen to alter biofilm formation and accretion was found to be dependent on growth conditions (FIG. 10). Whereas little change in biofilm was noted when modified M9 was used, supplementation of King's A medium with phytoglycogen reduced final biomass values by up to 53%.

As observed, concentrations of up to 10 mg phytoglycogen.ml⁻¹ modified M9 medium resulted in only small reductions in biofilm formed (FIG. 10). However, when the concentration of phytoglycogen was increased to 100 mg phytoglycogen.ml⁻¹, there was a substantial reduction in biofilm formation (modified M9 medium; FIG. 9). This was supported by quantitative measurements which demonstrated that supplementation with 100 mg phytoglycogen.ml⁻¹ medium resulted in biofilms with accreted biomass values which were 17.7%±3.3 (modified M9 medium) and 59.0%±8.0 (King's A medium) of the values for the respective biofilm grown in non-supplemented medium (n=14±SEM). The % reduction for King's A medium was similar at both 10 and 100 mg phytoglycogen.ml⁻¹, indicating that this may be the extent of efficacy in this medium.

That is, native phytoglycogen can limit both initial formation processes and subsequent biofilm growth. Furthermore, this ability is dictated in part by the local environmental conditions, as shown here by changing growth conditions, and is also concentration dependent with higher concentrations resulting in greater reductions. Similar to reports on the ability of other polysaccharides such as dextran to limit surface colonization and biofilm formation, native unmodified phytoglycogen impairs the ability of P. aeruginosa to form biofilms. This may occur at any, or all, of the steps of biofilm formation and growth, including cell attachment and adhesion, cell division and microcolony formation, microcolony expansion and maturation of a biofilm, and biofilm dispersal. P. aeruginosa is considered the model organism for biofilm studies and it is expected that the data obtained will have application to other organisms.

### Example 22

### Cationized Phytoglycogen is More Effective than Phytoglycogen in Inhibiting Biofilm Formation and Development

Example 21 relayed knowledge on the use of native and unmodified phytoglycogen to impede the ability of the model organism for biofilm studies, P. aeruginosa, to form biofilms. In this example, we utilise a cationized form of phytoglycogen.

P. aeruginosa PAO1 stocks were revived by sub-culturing P. aeruginosa PAO1 in modified

M9 or King's A medium (22-24 h, 37° C., 150 rpm); recipes and preparation are described in Example 21. The experiment was essentially performed as described in Example 21, with the exception that media were supplemented with sterile cationized phytoglycogen. Experiments were performed in triplicate assay and repeated as independent triplicate experiments (n=9).

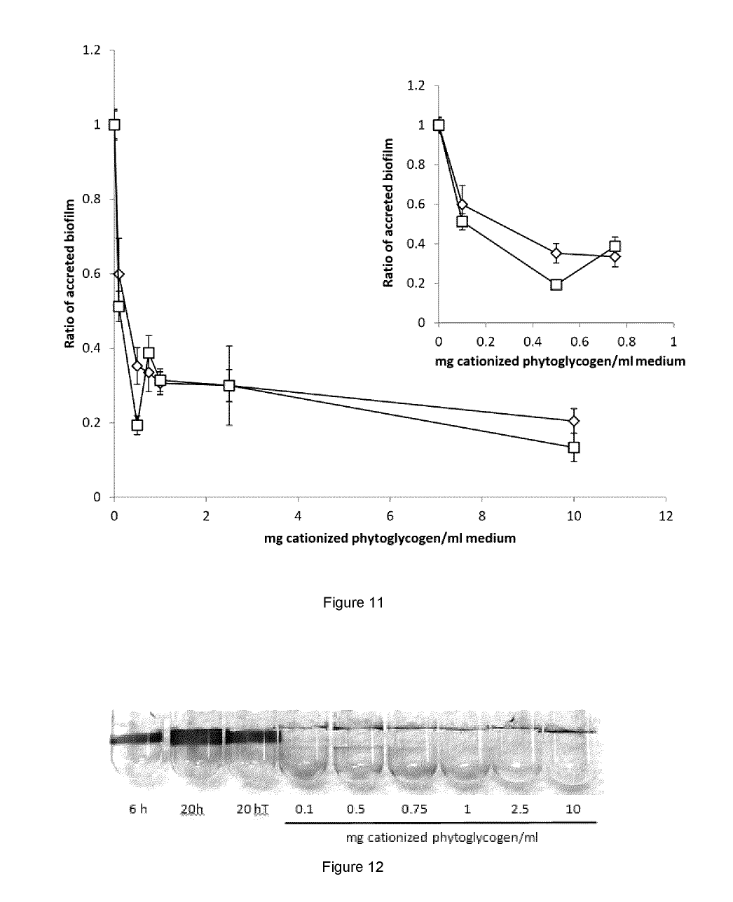

Growth in the presence of cationized phytoglycogen resulted in readily apparent visual and quantitative differences in the amount of biofilm formed. FIG. 9 shows an image of representative stained biofilms formed by P. aeruginosa grown with varying concentrations of cationized phytoglycogen. FIG. 11 shows that supplementation with increasing amounts of cationized phytoglycogen caused a steady reduction in accreted biofilm. Two different media were assessed with similar outcomes (open squares represent modified M9 medium and open diamonds represent King's A Medium).

While native phytoglycogen reduced biofilm growth (Example 21), lower concentrations of cationized phytoglycogen were required to bring about comparable biofilm prevention. Visual observations were supported by quantitative measurements as shown in FIGS. 10 and 11.

Cationized phytoglycogen possesses superior ability to native phytoglycogen in limiting biofilm accretion. Approximately ten-fold more native phytoglycogen is required to cause reductions similar to cationized phytoglycogen. In this respect, native and modified forms of phytoglycogen may serve as anti-biofilm and anti-fouling agents, or as part of a formulation.

### Example 23

### Cationized Phytoglycogen Limits Cell Attachment to Surfaces

The defining step of biofilm formation begins with the attachment of a cell to a surface. This is a multifactorial process, the outcome of which is determined by parameters such as cell, cell phenotype, cell surface properties and appendages, the properties of the surface and local environmental conditions. The ability to interfere with cell attachment to a surface is desirable since it will limit downstream biofilm growth.

P. aeruginosa PAO1 stocks were revived by sub-culturing P. aeruginosa PAO1 in Mueller-Hinton broth (Oxoid; Fisher Scientific, Canada) and grown for 20-22 h (37° C., 150 rpm). Cell attachment assay was done in sterile 96 well polystyrene plates. Briefly, stocks of broth, non-supplemented or supplemented with 1 or 10 mg cationized phytoglycogen.ml⁻¹, were inoculated to a final cell density of 5×10⁵ CFU.ml⁻¹.100 μl aliquots were pipetted into 8 wells/condition. Negative growth controls contained uninoculated media. Plates were incubated statically for 5 h at 37° C. and were then transferred to a biological safety cabinet. Well contents were aspirated using a pipette, discarded, and the wells rinsed with 0.9% (wt/vol) NaCl. Adhered cells were stained with 0.1% Hucker's crystal violet (100 μl/well) for 15 min at room temperature. Well contents were aspirated and the wells were washed excessively with water to remove any unbound stain. Plates were allowed to air dry. Retained stained was solubilised with 33% acetic acid (vol/vol; 100 μl/well) and then quantitated at A=600 nm using a BioTek EL800 plate reader. Experiments were done in octuplicate replicate and repeated three times (n=24±sem).

After a 5 h incubation period, P. aeruginosa cells incubated in the presence of non-supplemented growth medium yielded attachment values, as indicated by stain retention absorbance value, of 1.023±0.049, whereas incubation in the presence of 1 or 10 mg cationized phytoglycogen.ml⁻¹ caused reductions and had values of 0.345±0.012 and 0.536±0.041, respectively.

A greater reduction for attachment of P. aeruginosa to polystyrene plates was seen at 1 mg cationized phytoglycogen.ml⁻¹ than for the higher concentration of 10 mg.ml⁻¹. The most probable explanation relates to a balance of the interactions arising between nanoparticles and cells, and also the nature of the surface material, with polystyrene being relatively more hydrophobic. In this instance, cationized phytoglycogen, bearing a net positive charge, will be expected to bind to both the hydrophobic polystyrene surface and to the negatively charged cells.

There likely is some optimum which results in maximum repulsion between cells and surface, and will be influenced by other parameters such as the composition and concentration of nanoparticles. It is recommended that for a given application the formulation be empirically established and will take into account any surfaces, environmental milieu, cells and composition and concentration of nanoparticles.

### Example 24

### Pre-Treatment of Surfaces with Cationized Phytoglycogen Limits Cell Attachment

While initial cell attachment is a defining moment in the biofilm formation, the properties of the surface are also of consequence. The naïve surface properties influence the formation of the so-called conditioning film, which simplistically consists of moieties adsorbed onto the surface from the local environment, and may include lipids and proteins. The conditioning film is thus critical since it contributes to the overall surface properties; one approach to limiting biofilms is through deliberate alteration of surface properties via a conditioning film which is repellent to attachment and adhesion.

P. aeruginosa PAO1 stocks were revived by sub-culturing P. aeruginosa PAO1 in Mueller-Hinton broth (Oxoid; Fisher Scientific, Canada) and grown for 20-22 h (37° C., 150 rpm). Cell attachment assay was done in sterile 96 well polystyrene plates. Briefly, pre-treatment of wells was done by pipetting into the corresponding wells 100 μl of broth, or broth supplemented with 1 or 10 mg cationized phytoglycogen.ml⁻¹. The plate was sealed using ParaFilm™, and incubated for 20 h at 4° C. After 20 h the plates were transferred to a biological safety cabinet, well contents were aspirated, discarded and the wells washed with 0.9% NaCl (wt/vol). At this point, cell attachment to pre-conditioned wells was done as described in Example 23. Experiments were done in octuplicate replicate and repeated three times (n=24±sem).

Bacterial attachment in wells which were pre-treated with non-supplemented medium or 1 mg cationized phytoglycogen.ml⁻¹, had similar absorbance values of 1.023±0.049 and 1.036±0.045 units respectively (Table 5). Pre-treatment of wells with 10 mg cationized phytoglycogen.ml⁻¹ however reduced bacterial attachment by approximately 75% (measured absorbance value of 0.251±0.016).

TABLE 5
Pre-treatment of substratum and addition of cationized phytoglycogen
to the attachment medium both reduce cell adhesion to a surface

| Attachment broth | Pre-treatment of surfaces | | |
|---|---|---|---|
| (mg cationized | (mg cationized phytoglycogen · ml⁻¹) | | |
| phytoglycogen · ml⁻¹) | 0 mg · ml⁻¹ | 1 mg · ml⁻¹ | 10 mg · ml⁻¹ |
| 0 | 1.023 ± 0.049 | 1.036 ± 0.045 | 0.251 ± 0.016 |
| 1 | 0.345 ± 0.012 | 0.299 ± 0.010 | 0.266 ± 0.009 |
| 10 | 0.536 ± 0.041 | 0.489 ± 0.034 | 0.396 ± 0.025 |

n = 24 ± sem;
values are absorbance units (λ = 600 nm).

It was further found that combining surface pre-treatment and incubation with a solution containing cationized phytoglycogen limited cell attachment (Table 5). There was a trend in efficacy where maximum reduction was found to occur with surface pre-treatment of 10 mg cationized phytoglycogen.ml⁻¹ and followed a trend of 0>1>10 mg cationized phytoglycogen.ml⁻¹ of attachment broth. In contrast to the null efficacy of the 1 mg cationized phytoglycogen.ml¹ surface pre-treatment, supplementation of the attachment broth restored the ability to prevent attachment and was slightly more effective than incubation with attachment broth alone.

### Example 25

### Cationized Phytoglycogen Disrupts Biofilm Maturation

P. aeruginosa PAO1 stocks were revived by sub-culturing P. aeruginosa PAO1 in modified M9 medium and grown for 22-24 h (37° C., 150 rpm). Modified M9 medium was prepared as described in Example 21. Sterile glass tubes (18×150 mm) were used for the biofilm formation assay. A stock solution was inoculated with the stationary phase culture (1:2000 dilution), mixed well and 2 mL aliquots were transferred to sterile tubes. The tubes were incubated for 6 h at 37° C. and 150 rpm. Prior to the 6 h transfer time point, stock sterile solutions of media or media supplemented with modified phytoglycogen were prepared. At 6 h, the culture was removed by aspiration with a pipette and immediately replaced with the appropriate pre-warmed solution. The tubes were returned to the incubator for a further 14 h for a total incubation time of 20 h at 37° C. and 150 rpm. Negative controls comprised non-transferred and transferred non-supplemented medium sample tubes and were used to assay for discrepancies arising from the transfer step. At 20 h, the accumulated biofilm was quantified as described in Example 14. Experiments were performed in triplicate assay and repeated as independent quadruplicate experiments (n=12).

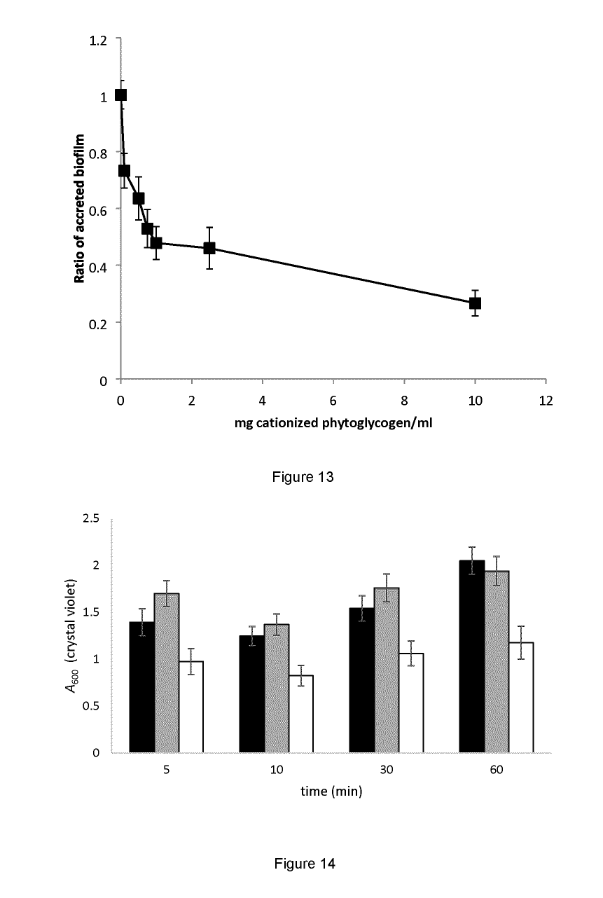

The treatment of 6 h-old biofilms with cationized phytoglycogen resulted in an alteration in the accretion pattern at 20 h. FIG. 12 shows recorded images of representative biofilms formed by P. aeruginosa following treatment with varying concentrations of cationized phytoglycogen; FIG. 13 describes quantification of accretion of pre-formed biofilms from P. aeruginosa following treatment with varying concentrations of cationized phytoglycogen. At 6 h considerable biofilm was formed, albeit less than at 20 h. The 20 h and 20hT biofilms represent samples where biofilm was allowed to accumulate for 20 h without exchanging medium for 20 h (not transferred), and where the associated culture medium was exchanged at 6 hrs and then allowed to incubate for a total of 20 h (20hT—transferred). In both instances, similar amounts of biofilm were present and indicated that the transfer step had not disrupted the normal progression of events. In contrast, all samples where medium had been exchanged with medium supplemented with varying concentrations of cationized phytoglycogen displayed losses in biofilm accreted by 20 h (FIGS. 12 and 13).

Cationized phytoglycogen can prevent the continued maturation of a nascent biofilm. This effect is concentration-dependent and increasing concentrations correlate with greater efficacy. This may occur through interactions between the charged nanoparticles and cells within the biofilm, or with components of the extracellular matrix. This cessation of maturation may also be related to limited motility of cells, affecting microcolony expansion and specialization. In Example 20, cationized phytoglycogen was shown to impede motility, which is important for the development of the hallmark 3-D architecture of biofilms and which is vital to the development of micro-environments and the creation of a plethora of phenotypes.

### Example 26

### Short-Term Treatment of Biofilms with Cationized Phytoglycogen Causes a Reduction in Biofilm Mass

Cationized phytoglycogen may interact with both the cells within biofilms, and also with components of the extracellular matrix. It then follows that cationized phytoglycogen, through such interactions, may disrupt biofilms. This has been demonstrated for other positively-charged species, such as metal cations, and for chelating agents, which remove or displace cations from biofilms with ensuing damage to the fine structure arrangement and organisation between cells and matrix.

P. aeruginosa PAO1 stocks were revived by sub-culturing P. aeruginosa PAO1 in modified M9 medium and grown for 22-24 h (37° C., 150 rpm). Modified M9 medium was prepared (Example 21). Sterile glass tubes (18×150 mm) were used for the biofilm formation assay. A stock solution was inoculated with the stationary phase culture (1:2000 dilution), mixed well and 2 ml aliquots were transferred to sterile tubes. The tubes were incubated for 20 h at 37° C. and 150 rpm. At 20 h, sets of tubes were transferred to a biological safety cabinet, culture was removed by aspiration and immediately replaced with the appropriate pre-warmed transfer solution containing 1 mg native or cationized phytoglycogen.ml⁻¹. Non-supplemented medium was used as the negative control. Tubes were returned to the incubator and sampled 5, 10, 30 and 60 min post-transfer. The tube contents were removed by aspiration and discarded. The tubes were gently rinsed with sterile 0.9% (wt/vol) NaCl to remove loosely attached cells and the remaining biofilm biomass was quantitated (Example 21). Experiments were done in triplicate and repeated four times (n=12).

A short-term exposure assessment was performed to evaluate the impact of supplementation with cationized phytoglycogen on biofilms formed by P. aeruginosa PAO1. This has potential applications including as an anti-biofilm agent for the treatment of biofouled abiotic or biotic surfaces. Biofilms were treated with native or cationized phytoglycogen for a period of 5, 10, 30 or 60 minutes (FIG. 14). No loss of biofilm occurred following brief incubation in medium or medium supplemented with native phytoglycogen (FIG. 14). In contrast, cationized phytoglycogen stimulated an average reduction of 40%. Notably, this reduction remained relatively constant throughout the entire assessed time period with (averages of 43, 40, 40, 39% reduction at 5, 10, 30 and 60 min, respectively, and indicated that this reduction was a rapid event and that extended incubation of up to one hour did not increase the measured loss of biomass.

This effect contrasts with the outcomes following treatment with cationized phytoglycogen as described in Examples 22 and 25. It is proposed that cationized phytoglycogen acts upon the stages of biofilm formation and development through different mechanisms, including but not limited to formation of a conditioning film on substrata, impeded motility, altered cell surface properties, interference with quorum sensing-based processes, interactions with biofilm extracellular matrix substances and cells within biofilms. This multi-stage targeting of biofilms renders cationized phytoglycogen versatile as an anti-biofilm or anti-fouling agent.

### Example 27

### Cationized Phytoglycogen Inhibits the Ability of Sub-MIC of Select Antibiotics to Stimulate Biofilm Growth

Antibiotics are agents used to prevent or limit microbial infections within a host. A number of parameters are critical to successful outcomes, including choice of antibiotic and antibiotic concentration. Upon administering an antibiotic to a patient, the concentration will rise to achieve a maximum and then decline as the antibiotic is cleared from the body. Treatment regimens seek to maintain a therapeutic concentration. It is not uncommon however for periods of time when the concentration of the antibiotic may be at sub-minimum inhibitory concentration (MIC) levels. Sub-MIC of antibiotics have been shown to provoke responses by bacterial cells; critically these include survival and persistence strategies. For example, sub-MIC aminoglycoside antibiotics stimulate increased biofilm by the opportunistic pathogen P. aeruginosa.

The potential for cationized phytoglycogen to limit enhanced biofilm formation at sub-MIC concentrations of the aminoglycoside tobramycin was assessed in sterile 96 well polystyrene plates. P. aeruginosa PAO1 stocks were revived by sub-culturing P. aeruginosa PAO1 in Mueller-Hinton broth (Oxoid; Fisher Scientific, Canada) and grown for 20-22 h (37° C., 150 rpm). Sterile solutions of gamma-irradiated cationized phytoglycogen was prepared by reconstitution and dilution as required in sterile Mueller-Hinton broth. Wells contained final concentrations 0, 0.25, 0.50, 0.75, 1.0, 1.25, 1.5 times the MIC value, which was established empirically as 0.4 μg tobramycin.ml⁻¹, in the absence or presence of 1 or 10 mg.ml⁻¹ cationized phytoglycogen. Negative growth controls comprised sterile medium, non-supplemented or with 1 or 10 mg cationized phytoglycogen.ml⁻¹. Positive growth controls contained inoculated medium, non-supplemented or with 1 or 10 mg cationized phytoglycogen.ml⁻¹. The inoculum was prepared immediately prior to use by diluting in sterile Mueller-Hinton broth, with a final in-assay cell density of 5×10⁵ CFU.ml⁻¹. Plates were incubated statically at 37° C. At 20 h, biofilms were stained and quantitated according to the previously described protocol (Example 23). Experiments were done in quadruplicate replicate and repeated three times (n=12).

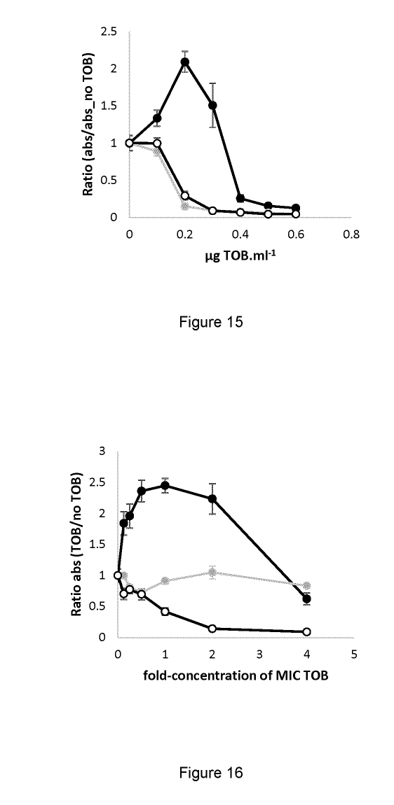

In agreement with the published literature, sub-MIC concentrations of the aminoglycoside tobramycin resulted in increased biofilm formation (FIG. 15). Relative to the medium only control, biofilm was found to accumulate by factors of 1.33 (at ¼-MIC), >2.00 (at ½-MIC) and 1.5 (at % -MIC). Only at values ≥MIC was biofilm accumulation reduced.

Data sets were also normalized relative to their corresponding value for biofilm accumulation when no tobramycin was added (FIG. 15). When medium was supplemented with cationized phytoglycogen, there was no increase in biofilm formed at ¼-MIC tobramycin and indicated inhibition of enhanced biofilm due to sub-MIC antibiotic. At ½-MIC tobramycin, cationized phytoglycogen reduced biofilms by ca. 70% relative to biofilm formed in the corresponding supplemented medium. At ¾ the MIC of tobramycin, this reduction was greater still, attaining levels of 90%. Similar effects were not seen in non-supplemented medium until concentrations of tobramycin had exceeded the MIC.

When cationized phytoglycogen is used in combination with an antibiotic which is below its therapeutic concentration, it reverses enhanced biofilm growth. Biofilms are an identified critical step in the development of acute and chronic infectious disease, and are also less tractable to chemotherapeutic regimens and protected from host immune defense and response capabilities. Combination therapy with cationized phytoglycogen thus represents a method to prevent enhanced biofilm formation when an antibiotic concentration is less than that which is therapeutically required to cause inhibition of cell growth or cell death. By preventing biofilm formation and proliferation, these microbial cells remain susceptible to the action of antibiotics, especially once the next dose is delivered. Additionally, since the microbial cells do not enter the sheltered state of a biofilm, these cells remain accessible to the protective actions of the host immune system.

### Example 28

### Combining Cationized Phytoglycogen and Antibiotic Enhances Biofilm Eradication

It has previously been shown that cationized phytoglycogen could interfere with cell motility, cell attachment, biofilm formation and maturation, and also render bacteria more susceptible to the action of diverse classes of antibiotics (Examples 17, 19, 20, 22-26). In addition, cationized phytoglycogen reversed the propensity of sub-MIC antibiotics to enhance biofilm proliferation (Example 27). We demonstrate that combining cationized phytoglycogen with antibiotics is an improved method for biofilm reduction and treatment.

Biofilm growth and treatments were performed in sterile 96 well polystyrene plates. Sterile solutions of gamma-irradiated cationized phytoglycogen was prepared by reconstitution and dilution as required in sterile Mueller-Hinton broth. P. aeruginosa PAO1 stocks were revived by sub-culturing P. aeruginosa PAO1 (37° C., 150 rpm, 16-18 h) in Mueller-Hinton broth (Oxoid; Fisher Scientific, Canada). The inoculum was prepared immediately prior to use by diluting in sterile Mueller-Hinton broth to a final in-assay cell density of 5×10⁵ CFU.ml⁻¹. 100 μl of this solution was transferred to wells and incubated (37° C., 150 rpm). Sterile broth was the negative growth control. At 20 h, plates were transferred to a biological safety cabinet, well contents were gently aspirated and the wells rinsed with sterile 0.9% (wt/vol) NaCl. The 20 h biofilms were then exposed to ciprofloxacin or tobramycin at 0, 0.125, 0.25, 0.5, 1, 2, and 4× MIC, in Mueller-Hinton broth with 0, 1 or 10 mg cationized phytoglycogen.ml⁻¹. MIC values were established empirically as 0.4 and 0.125 μg.ml⁻¹ for tobramycin and ciprofloxacin respectively. Negative growth controls comprised sterile medium, non-supplemented or with 1 or 10 mg cationized phytoglycogen.ml⁻¹ in wells which had previously contained sterile growth medium. Positive growth controls contained inoculated medium, non-supplemented or with 1 or 10 mg cationized phytoglycogen.ml⁻¹. Plates were incubated at 37° C. for a further 24 h, after which biofilms were quantitated as previously described. Experiments were done in quadruplicate replicate and repeated three times (n=12).

Of the two antibiotics selected, cationized phytoglycogen sensitized cells to the action of ciprofloxacin but not tobramycin (Example 17). Biofilms were grown for 20 h prior to treatment for 24 h with different combinations of antibiotic and cationized phytoglycogen (FIG. 16, 17).

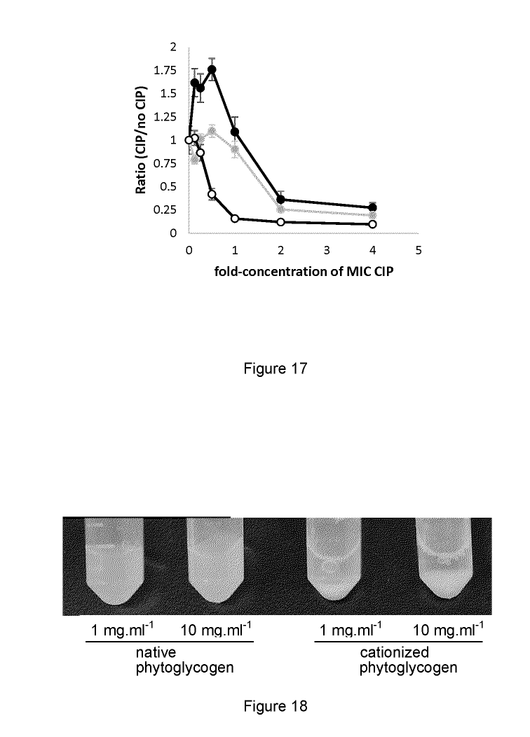

When 20 h biofilms were treated with tobramycin, similar to induction of biofilm formation by sub-MIC tobramycin, there was a net increase in biofilm proliferation for the range 0.125 to 2 times the MIC value (FIG. 16). At four-fold the MIC of tobramycin, biofilms were reduced by 40% relative to the initial biofilm. Whilst it is known that biofilms are less susceptible to antibiotics, the data indicated antibiotics may stimulate biofilm growth. A similar and unanticipated trend was also found for sub-MIC ciprofloxacin (FIG. 17). There was no change in biofilm at the MIC, and reductions of 64% and 73% at two- and four-fold the MIC values, respectively.

Treatment of biofilms with either tobramycin or ciprofloxacin in combination with 1 mg cationized phytoglycogen.ml⁻¹ was sufficient to prevent antibiotic-induced increases in biofilm accumulation (FIGS. 16, 17). Supplementation with 10 mg cationized phytoglycogen.ml⁻¹ resulted in reductions in biofilm accumulation at sub-MIC concentrations for both tobramycin and ciprofloxacin (FIGS. 16, 17). For example, at the MIC of tobramycin, this results in biofilms which are approximately 10% of the corresponding MIC tobramycin in medium alone control. At 0.5 MIC of ciprofloxacin, the biofilm was 20% of the corresponding 0.5 MIC in medium alone.

In summary, when cationized phytoglycogen is used in combination with an antibiotic, it reverses the extent of antibiotic-induced biofilm proliferation. Biofilms are an identified critical step in the development of acute and chronic infectious disease, and are also less tractable to chemotherapeutic regimens and protected from host immune defense and response capabilities. Combination therapy with cationized phytoglycogen thus represents a method to prevent the enhanced biofilm growth attributable to antibiotics when these are at concentrations which are below those therapeutically required to cause inhibition of cell growth or cell death. When a higher concentration of cationized phytoglycogen is used in combination with antibiotic, this reduced the amount of biofilm.

### Example 29

### Cationized Phytoglycogen Causes Cells to Sediment from Suspension

Sedimentation of cells from a cell suspension may occur through a number of mechanisms including flocculation or a reduction in overall surface charge resulting in reduced repulsion between particles. Sedimentation of fine particles is important for processes such as water purification and treatment. In addition, through altering the interactions between cells in solution, or cells and a substratum, the progression of colonization, attachment and biofilm formation may be affected.

Sterile solutions of gamma-irradiated cationized phytoglycogen was prepared by reconstitution and dilution as required in sterile Mueller-Hinton broth. P. aeruginosa PAO1 stocks were revived by sub-culturing P. aeruginosa PAO1 (37° C., 150 rpm, 16-18 h) in Mueller-Hinton broth (Oxoid; Fisher Scientific, Canada). 1 ml aliquots of cells were centrifuged for 5 min at 6000×g, washed twice with 5 mM HEPES buffer (pH 6.8), and resuspended in HEPES buffer containing 0, 1, or 10 mg native or cationized phytoglycogen.ml⁻¹. The samples were then placed on the bench top for 30 min, after which observations were made on sediment formation. Whole mount negatively-stained preparations of samples were observed using transmission electron microscopy. The high concentration of background particles was reduced by pelleting cells briefly (3000×g, 5 min), the supernatant removed and the pellet gently resuspended in 5 mM HEPES buffer (pH 6.8). A Formvar- and carbon-coated copper grid (200 mesh; Marivac) was floated, film side down, on 10 μl of sample for 10 s. The grid was removed and the edge touched to Whatman no. 1 filter paper to wick off excess. The grid was then washed by floating (sample side) on 50 μl of nanopure water, blotted, floated on 10 μl of 2% (wt/vol) uranyl acetate for 10 s, and blotted dry. Grids were examined using a Philips CM10 transmission electron microscope operating at an acceleration voltage of 80 kV under standard operating conditions.

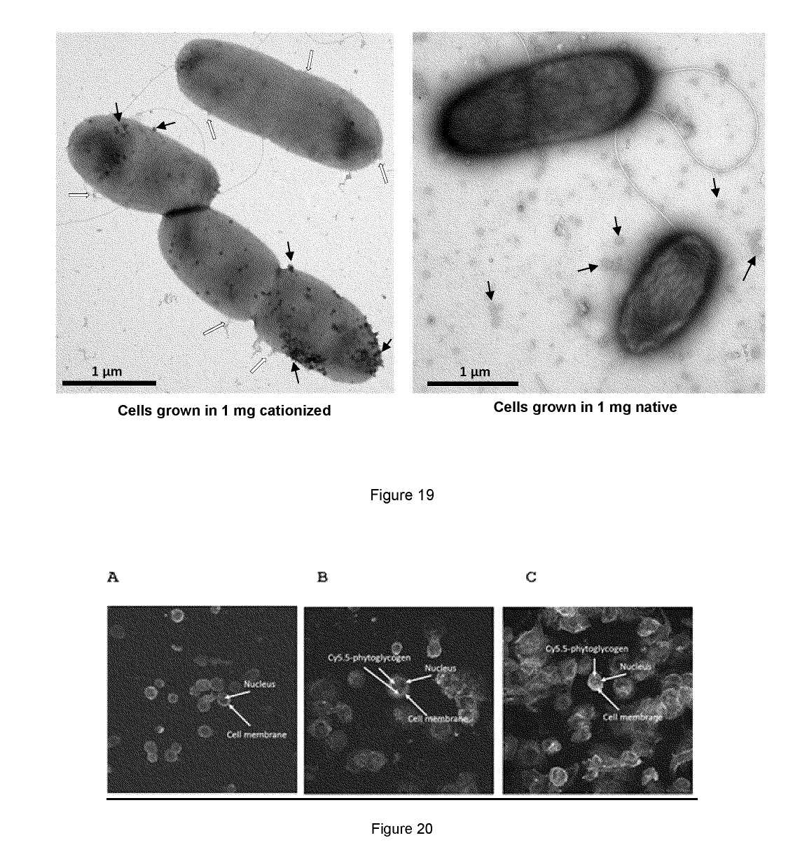

It was found that supplementation with cationized phytoglycogen but not native phytoglycogen caused P. aeruginosa cells to sediment out of solution (FIG. 18). This was a rapid process, with sediment visible within 10 min. Microscopy of the cells indicated that cationized but not native phytoglycogen interacted directly with the cells (FIG. 19). It is expected that this could cause changes to the overall cell surface charge, resulting in sedimentation of the cells. In addition, this would interfere with processes such as attachment and biofilm formation.

It was also noticed that cells treated with cationized phytoglycogen showed indications of perturbation of the cell wall (FIG. 19). This may in part explain the sensitization to antibiotics by cationized phytoglycogen (Example 17), and could also cause changes in membrane parameters which may be significant to diffusion processes such as uptake of antibiotics, or quorum sensing signals.

### Example 30

### Skin Irritation Potential of Phytoglycogen Nanoparticles

Human Repeat Insult Test (HRIPT) was carried out to evaluate the cutaneous irritation (contact dermatitis) and sensitization potential (contact allergy) of the cream formulations containing phytoglycogen over a 3-weeks period followed by a rest period and a challenge period. The study was designed as a single center, randomized study; double-blinded with interindividual comparison of treatments between the test formulations: cream base with and without phytoglycogen. The formulation of the cream base with phytoglycogen is described in Example 31. In the case of cream base without phytoglycogen the later was replaced with corresponding amount of water.

A total of 50 healthy volunteers of either sex between 24 and 76 years old (Average Age=46.88) were chosen for this study.

Patches containing the test formulations and a control (Pure Vaseline USP) were applied to the test area and left in contact with the skin. The first patch was removed after 48 hrs and following the examination, a new patch with fresh test formulation was applied. The test formulations were applied on the selected zones every second day, three times per week, over 3 consecutive weeks. This is referred as the Induction Phase.

After the completion of the Induction Phase described above, a Rest Period of 10-14 days was scheduled. Then the Challenge Phase was commenced. For this phase, the patch was applied to a different site than the one which was used in the Induction Phase. After 48 hrs of application the patch was removed, the test site was cleaned and examined by a dermatologist for any signs of intolerance or irritation.

The results of HRIPT for the formulation containing phytoglycogen summarized in Table 6.

TABLE 6
Dermatological Investigation

| | | | | | | | | | | Induction | |
|---|---|---|---|---|---|---|---|---|---|---|---|
| | | | | | | | | | | Period | |
| | Induction Period Observations | | | | | | | | | Observations | |
| | #1 | #2 | #3 | #4 | #5 | #6 | #7 | #8 | #9 | 48 h | 72 h |
| Skin Reaction* | % of subjects with observed reaction | | | | | | | | | | |
| 0 | 98 | 100 | 100 | 100 | 100 | 100 | 100 | 100 | 100 | 100 | 100 |
| + | 2 | 0 | 0 | 0 | 0 | 0 | 0 | 0 | 0 | 0 | 0 |
| 1 | 0 | 0 | 0 | 0 | 0 | 0 | 0 | 0 | 0 | 0 | 0 |
| 2 | 0 | 0 | 0 | 0 | 0 | 0 | 0 | 0 | 0 | 0 | 0 |
| 3 | 0 | 0 | 0 | 0 | 0 | 0 | 0 | 0 | 0 | 0 | 0 |
| 4 | 0 | 0 | 0 | 0 | 0 | 0 | 0 | 0 | 0 | 0 | 0 |

*0 = No visible reaction
+ = Erythema barely noticeable
1 = Mild (slight) erythema
2 = Moderate but well defined erythema and presence of slight oedema
3 = Marked erythema, presence of oedema and vesicles
4 = Severe erythema, presence of vesicles, blisters, and ulcerations

Based on the results of the study described herein, it was concluded that phytoglycogen produced no signs of cutaneous irritation nor skin sensitization. It is therefore considered non-irritant and hypo-allergenic substance.

### Example 31

### A Pharmaceutical Composition Comprising Phytoglycogen

This preparation under the form of a cream contains phytoglycogen as active ingredient and is more suitable for a topical administration.

The formula of the cream with phytoglycogen is described in Table 7.

TABLE 7
Pharmaceutical composition comprising phytoglycogen

| Phase | Ingredient Name | % |
|---|---|---|
| A | Aqua | 71.0 |
| A | Glycerin | 5.0 |
| A | Xanthan Gum | 0.6 |
| A | Phytoglycogen | 2.0 |
| B | Almond Oil | 5.0 |
| B | Avocado Oil | 5.0 |
| B | Butyrospermum Parkii (Shea | 4.0 |
| | Butter) Fruit | |
| B | Cetyl Alcohol | 2.0 |
| B | Dimethicone | 2.0 |
| B | Stearyl Alcohol | 1.0 |
| B | Sorbitan Stearate | 1.4 |
| C | Phenoxyethanol, sorbic acid, | 0.8 |
| | caprylic glyceryl | |
| C | Tocopherol acetate | 0.2 |
| C | Triethanolamine | * |

* added dropwise until pH reaches 5.5

The composition was prepared as follows:

Water and glycerin were combined in a beaker. Then phytoglycogen and xanthan gum were dispersed. The mixture was heated to 75° C. Phase B ingredients were combined in a separate beaker, and heated to 75° C. Phase B was added to phase A and mixed at 1200 rpm until homogeneous. Phase C ingredients were added to the mixture when the temperature has decreased to 40° C. The mixing continued at 400 rpm for 10 minutes to ensure thorough mixing.

### Example 32

### Internalization of Cy5.5-Labeled Glycogen/Phytoglycogen Particles by TCP-1 Monocytes

Cy5.5-labeled glycogen/phytoglycogen particles were produced as described in Example 10. Conjugation of a near-infrared fluorescent dye (Cy5.5) to the particles used in this study enabled analysis of nanoparticle uptake by confocal fluorescence microscopy.

MCP-1 cells were incubated with Cy5.5-labeled glycogen/phytoglycogen particles at a concentration of 1 mg/mL at 4° C. (negative control) and 37° C. for 0.5, 2, 6 and 24 hrs. Then cells were washed with PBS, fixed in 10% Buffered Formalin Solution and washed again with PBS. Than fixed cells were stained with DAPI (nucleus) and AF488 (cell membrane). Internalization of glycogen/phytoglycogen particles was assessed by Olympus Fluoview FV1000 Laser Scanning Confocal Microscope.

Incubation at 4° C. when endocytotic and phagocytotic processes are no longer active did not result in any particles associated with THP-1 cells (FIG. 20). This confirmed that there was no accumulation of the nanoparticles by THP-1 cells due to the surface binding. In contrast, incubation at 37° C. for over 6 hrs revealed considerable accumulation of Cy5.5-labeled glycogen/phytoglycogen particles in cell cytoplasm (FIG. 20). However, there was very low uptake in the time interval of 0.5-2 hrs.

### Example 33

### Pharmacokinetic (PK) Profile in Naive Mouse After Injection of Cy5.5-Phytoglycogen Conjugate

Cy5.5 labeled phytoglycogen (0.08 μM Cy5.5/mg) was synthesized as described in Example 10.

Nude CD-1 mice (n=3), 18-20 grams were injected with Cy5.5-Phytoglycogen dispersed in

PBS at a dose of 300 mg/kg mice. Small blood samples (50 μl) were collected from the mouse (submandibular vein) using heparinized tubes at multiple time intervals (15 mins, 1 hr, 2 hrs, 6 hrs and 24 hrs). These time points were analyzed by fluorescence using a cytofluorimeter plate reader. Nanoparticle concentration was interpolated using a standard curve consisting of known concentrations of Cy5.5-phytoglycogen nanoparticle diluted in blood.

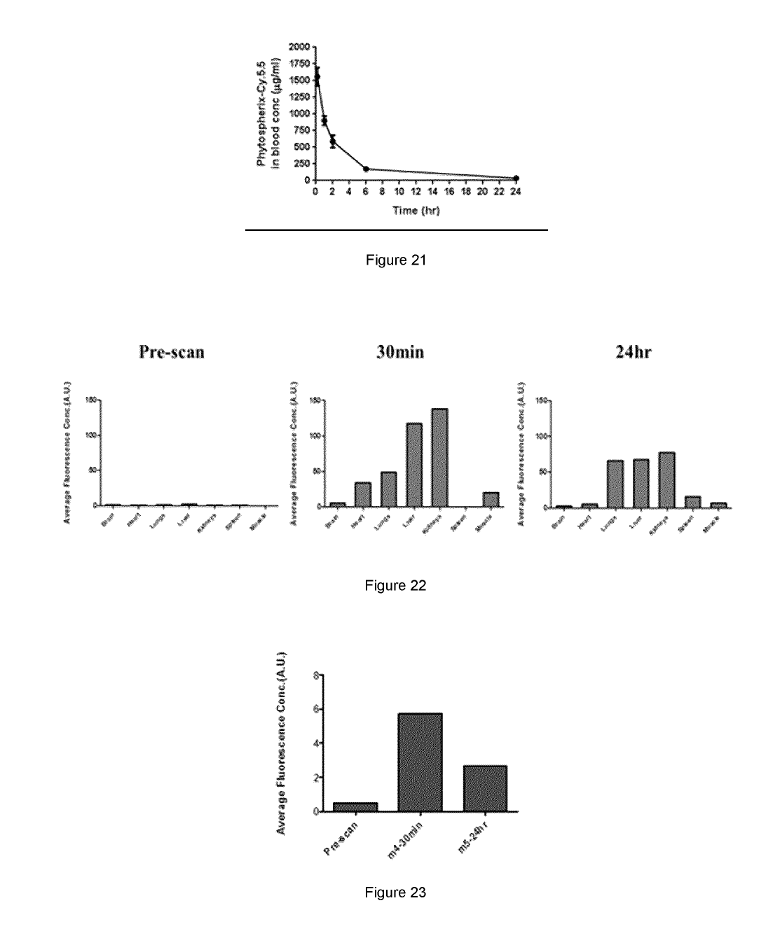

As can be seen from FIG. 21, Cy5.5-phytoglycogen concentration in blood decreased over the time in exponential manner and was eliminated by 24 hrs. The elimination half-life was determined (calculated) to be 2 hrs. Half-life refers to the period of time required by the body to reduce the initial blood concentration of the compound by 50%.

All optical imaging experiments were performed using a small-animal time-domain eXplore Optix MX2 pre-clinical imager, and images were analyzed or reconstructed as fluorescence concentration maps using ART Optix Optiview analysis software 2.0 (Advanced Research Technologies, Montreal, QC). A 670-nm pulsed laser diode at a repetition frequency of 80 MHz and a time resolution of 12 ps light pulse was used for excitation. The fluorescence emission at 700 nm was collected by a highly sensitive time-correlated single photon counting system and detected through a fast photomultiplier tube.

Cy5.5 labeled phytoglycogen (0.8 μM Cy5.5/mg) was synthesized as described in Example 10.

In naïve animals, in vivo imaging revealed strong signals of Cy5.5-Phytoglycogen in liver, lungs (at all time points), kidney (15 min-6 h), bladder (15 min-6 h), and brain (15 min-2 hrs) (FIGS. 22 and 23).

Ex vivo data at 30 min and 24 h confirmed that indeed there was significant uptake of the Cy5.5-Phytoglycogen in lungs and to a lesser degree in brain (FIGS. 22 and 23). The signal in the brain was highest at earlier time points (30 min) compared to later time points (24 h). Since the Cy5.5-Phytoglycogen nanoparticle is a glucose polymer, it is possible that organs such as brain and lungs, known to be very active in glucose transport, accumulate Cy5.5-Phytoglycogen via glues transporters.

The in vivo imaging data demonstrated that the liver is mainly responsible for metabolism of the Cy5.5-Phytoglycogen. Furthermore, it is possible that metabolized in liver nanoparticles produce smaller Cy5.5-labeled glucose derivatives that can re-enter the blood stream and then be eliminated through the renal system.

1. An anti-infective composition comprising nanoparticles, each nanoparticle comprising a glycogen or phytoglycogen polymer functionalized with an anti-infective component, wherein the anti-infective component comprises one or more molecules that imparts anti-infective activity to the nanoparticle, and a carrier.

2. The anti-infective composition of claim 1, wherein the nanoparticles have a PDI of less than about 0.3 as measured by dynamic light scattering and an average particle diameter of between about 30 nm and about 150 nm, about 60 to about 110 nm about 40 nm and about 140 nm, about 50 nm and about 130 nm, about 60 nm and about 120 nm, about 70 nm and about 110 nm, about 80 nm and about 100 nm, about 30 nm and about 40 nm, about 40 nm and about 50 nm, about 50 nm and about 60 nm, about 60 nm and about 70 nm, about 70 nm and about 80 nm, about 80 nm and about 90 nm, about 90 nm and about 100 nm, about 100 nm and about 110 nm, about 110 nm and about 120 nm, about 120 nm and about 130 nm, about 130 nm and about 140 nm, or about 140 nm and about 150 nm.

3-4. (canceled)

5. The anti-infective composition of claim 1, wherein the anti-infective component comprises an antibiotic, an antifungal or anti-protozoal compound.

6. (canceled)

7. The anti-infective composition of claim 1, wherein the anti-infective component comprises positively charged molecules bound to the surface of the glycogen or phytoglycogen nanoparticles.

8. The anti-infective composition of claim 7, wherein the nanoparticles are functionalized with a primary, secondary, tertiary or quaternary ammonium compound.

9. (canceled)

10. The anti-infective composition of any one of claim 8, wherein the nanoparticles are functionalized with a C₂ to C₃₂ quaternary ammonium compound.

11. The anti-infective composition of claim 1, wherein the nanoparticles are further functionalized with a hydrophobic functional group.

12. The anti-infective composition of claim 1, wherein the composition is for topical administration.

13. The anti-infective composition of claim 7, wherein the nanoparticles are further conjugated to one or more pharmaceutical or diagnostic agents.

14-15. (canceled)

16. An implant or a biomedical device or a coating for an implant or biomedical device comprising the anti-infective composition according to claim 1.

17-19. (canceled)

20. The anti-infective composition according to claim 1, wherein the composition is dissolved in a water-based or alcohol-based solvent, and wherein the nanoparticles are present at a concentration of 0.1-20% by total weight.

21. A method of treating an infection comprising administering a therapeutically effective amount of the composition of claim 1 to a subject in need thereof.

22. (canceled)

23. The method of claim 21 wherein the microbial infection is a bacterial infection.

24. The method of claim 23 wherein the bacterial infection is caused by P. aeruginosa.

25. The method of claim 24, wherein the patient has cystic fibrosis.

26-27. (canceled)

28. The method of claim 21 wherein the infection is an intracellular infection.

29. A skin sanitizer comprising the anti-infective composition according to claim 1.

30. The skin sanitizer according to claim 29, comprising:
about 25% to about 75%, by total weight, of a disinfecting alcohol;
about 0.1%-5.0%, by total weight, the anti-infective composition;
a virucidally effective amount of an organic acid; and
water.

31. (canceled)

32. A surface sanitizer comprising the anti-infective composition according to claim 1

33. The surface sanitizer according to claim 32, comprising:
about 50% to 90%, by total weight, of a disinfecting alcohol;
about 0.5%-10.0%, by total weight, the anti-infective composition;
an acid component sufficient to maintain the pH of the composition below about 5 which constitutes about 0.1% to about 5%, by total weight; and
water.

34. (canceled)

35. A method of inhibiting biofilm formation on a surface comprising applying an anti-infective composition of claim 1 to the surface.

36-46. (canceled)
